(19) United States
(12) Patent Application Publication
(10) Pub. No.: US 2017/0285032 A1
(43) Pub. Date: Oct. 5, 2017

(54) COMPOSITIONS AND METHODS FOR DIAGNOSIS AND PROGNOSIS OF COLORECTAL CANCER

(71) Applicant: Onconome, Inc., Redmond, WA (US)

(72) Inventor: Eva I-Wei AW, Shoreline, WA (US)

(21) Appl. No.: 15/487,952

(22) Filed: Apr. 14, 2017

Related U.S. Application Data
(63) Continuation of application No. 13/148,881, filed on Aug. 10, 2011, now abandoned.
(60) Provisional application No. 61/154,303, filed on Feb. 20, 2009.

(51) Int. Cl. G01N 33/574

(52) U.S. Cl. CPC G01N 33/57419

(57) ABSTRACT

Certain embodiments of the present invention provide methods and compositions related to the detection of colorectal cancer based upon the identification of biomarkers and combinations of biomarkers that indicate the present of colorectal cancer. One embodiment of the present invention provides a method for detecting colorectal cancer in a subject by obtaining a biological sample from the subject; detecting one or more biomarkers present in the sample; and comparing the concentrations and/or expression levels of the one or more biomarkers within the biological sample with the concentrations and/or expression levels of the one or more biomarkers in a normal control sample.

### CROSS REFERENCE TO RELATED APPLICATION

This application claims the benefit of Provisional Application No. 61/154,303, filed Feb. 20, 2009.

### TECHNICAL FIELD

The present invention relates to methods of diagnosing colorectal cancer and, in particular, to the use of a panel of biomarkers in the diagnosis of colorectal cancer from a biological sample.

### BACKGROUND

Colorectal cancer (CRC) is the third most prevalent malignancy in the United States with approximately 145,000 new diagnoses and 56,000 deaths estimated for 2005. Despite advances made, the efficacy of therapy has reached a plateau, making early diagnosis fundamental to reduce morbidity and mortality, especially in view of the fact that patients diagnosed at early stages show long-term survival. Stage I patients have a survival rate of ˜85%, while the 5-year survival rate drops to ˜65-75% in stage II patients and to 35-50% in stage III patients.

The most common non-invasive test for colorectal cancer is the fecal occult blood test (“FOBT”). Unfortunately, in addition to its high false-positive rate, the sensitivity of the FOBT remains around 50% and may not detect early malignancy, since not all carcinomas shed blood, Numerous serum markers, such as carcinoembryonic antigen (“CEA”), carbohydrate antigen 19-9, and lipid-associated sialic acid, have been investigated in colorectal cancer, but their low sensitivity has induced the American Society of Clinical Oncology to state that none can be recommended for screening and diagnosis, and that their use should be limited to postsurgery surveillance. Colonoscopy and sigmoidoscopy remain the gold standard for detecting colon cancer. These invasive exams are expensive, require highly trained staff, are uncomfortable, and raise the risk of bowel perforation and possible mortality. In addition, the normal sterilization process for endoscope, while effective against bacteria and many viruses, may not be effective against prions, and thus colonoscopy potentially expose patients to prion infection. Consequently, there is still a great need for new biomarkers and diagnostic tests for colorectal cancer. Since the treatment tor colorectal cancer is very much stage-dependent, clinicians, researchers, and various additional medical personnel and, ultimately, medical patients, all continue to seek a diagnostic tool for colorectal-cancer-stage identification.

### SUMMARY

Certain embodiments of the present invention provide methods and compositions related to the detection of colorectal cancer based upon the identification of biomarkers and combinations of biomarkers that indicate the present of colorectal cancer. One embodiment of the present invention provides a method for detecting colorectal cancer in a subject by obtaining a biological sample from the subject; detecting one or more biomarkers present in the sample; and comparing the concentrations and/or expression levels of the one or more biomarkers within the biological sample with the concentrations and/or expression levels of the one or more biomarkers in a normal control sample.

### BRIEF DESCRIPTION OF THE DRAWINGS

The following Detailed Description, given by way of examples, but not intended to limit the invention to specific embodiments described, may be understood in conjunction with the accompanying figures, in which:

FIG. 1 shows an SDS-PAGE gel image of 14 serum samples.

FIGS. 2A and 2B show western blots for colorectal cancer and normal serum samples probed with 1 μg/ml rabbit polyclonal anti human fibronectin antibodies and 1 μg/ml of rabbit IgG1 isotype antibodies as a negative control

FIG. 3 shows representative selected-reaction-monitoring mass spectrometry (“SRM-MS”) chromatograms for α-1-acid glycoprotein 1 (“ORM 1”) working peptides.

FIG. 4 shows representative multiple-reaction-monitoring mass spectrometry (“MRM-MS”) peptide trend lines of three ORM1 working peptides for 33serum samples.

FIG. 5 shows boxplots of cancer vs. normal for serum values for amyloid A protein (“SAA2”), ORM1, plasma serine protease inhibitor (“SERPINA3”), and C9 complement component (“C9”).

FIG. 6 shows a matrix plot for SERPINA3, ORM1, SAA2, and C9.

FIG. 7 shows a hierarchical clustering analysis of plasma and serum samples.

FIG. 8 shows an MRM-MS C9 tread line for three transition peptides of the C9 protein.

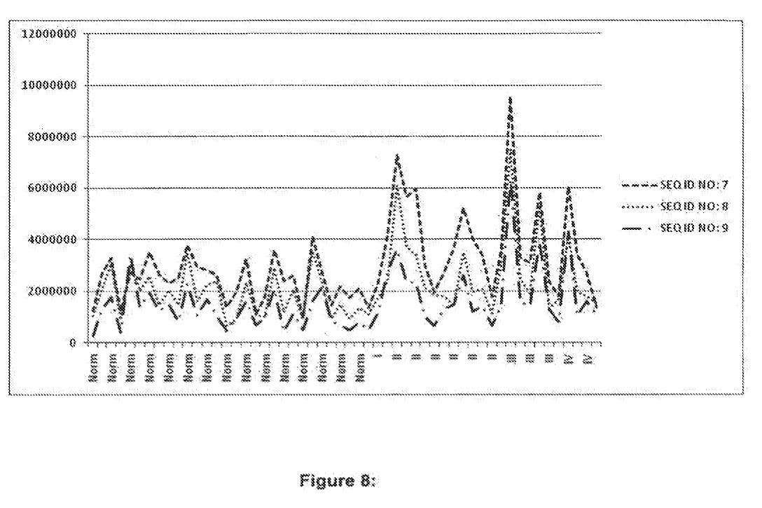

FIG. 9 shows a Receiver Operating Characteristic (“ROC”) curve for a 48-serum-sample set using the random-forest model.

FIG. 10 shows an ROC curve for 48 serum samples using the boosting method.

FIG. 11 shows an ROC curve for a 33-serum-sample set constructed by the random-forest model.

FIG. 12 shows an ROC curve for 13-serum-sample set constructed by the random-forest model.

### DETAILED DESCRIPTION

Certain embodiments of the present invention are described below, in overview, followed with experimental examples. Two appendixes with sequences listing are provided following the detailed description.

Mass spectrometry-based strategies for protein identification and quantification have made it possible to perform global, large scale comparative proteomic analysis of complex biological samples. Specifically, multiple-reaction-monitoring mass spectrometry (“MRM-MS”), a state-of-art mass spectrometry mode, offers very high sensitivity and speed for the identification and quantification of specific peptides in complex biological mixtures, and thus has promise for high-throughput screening of conical samples for candidate markers. MRM-MS has been well established in the pharmaceutical industry for small-molecule defection and in clinical laboratories for analysis of drug metabolites. Given the large number of potential biomarkers for disease detection, conventional diagnostic tools such as ELISA, which require expensive reagents and long development times, are not generally suitable for proteomic analysis. A portable MRM-MS assay, which provides low cost and fast turn-around time, is an attractive choice as the next generation assay platform in clinical laboratories, and is a promising basis for developing a diagnostic tool for colorectal-cancer-stage identification.

Certain embodiments of the present invention are based, in part, on the identification of a panel of biomarkers that are associated with colorectal cancer. These biomarkers are listed in Table 1. These biomarkers are present at different levels in the biological samples of colorectal cancer patients than in normal control samples. Accordingly, certain embodiments of the present invention relates to methods for the diagnosis, prognosis, and monitoring of colorectal cancer, including the different stages of colorectal cancer, by detecting or determining, in a biological sample obtained from a subject, the presence of an amount or level of at least one biomarker identified in Table 1. In particular embodiments of the present inventions the presence, an amount, and/or a level of at least two biomarkers, at least three biomarkers, at least four biomarkers, at least five biomarkers, at least six biomarkers, at least 7 biomarker, at least 8 biomarkers, etc. (including at least 9, 10, 11, 12, 13, 14, 15, 16, 17, 18, 19, 20, or more biomarkers, in any combination) of the biomarkers listed in Table 1 are determined. In alternative embodiments of the present invention, the amounts or levels of additional biomarkers, not listed in Table 1, may also be determined, including carcinoembryonic antigen (“CEA”), carbohydrate antigen 19-9, and, in still further embodiments of the present invention, to detect different types of cancer or other pathologies.

The term “biological sample” refers to any biological sample obtained from a human subject including, e.g., a tissue sample, a cell sample, a tumor sample, and a biological fluid such as blood, serum, plasma, or urine. In one embodiment of the present invention, the biological sample is serum.

A “biomarker” is a molecule produced by a cell or a tissue in an organism whose presence, level of expression, or form is correlated with cancer, e.g., colorectal cancer. Such molecules include nucleic acids, oligonucleotides, polynucleotides, peptides, polypeptides, and proteins, including polynucleotides, peptides, polypeptides, and proteins modified by the addition of polysaccharides, lipids, and various small molecules and functional groups.

### Methods for Diagnosis

In certain embodiments of the present invention, the presence of colorectal cancer in a subject may be detected by a difference between the expression levels of one or more of the selected biomarkers of Table 1 in a biological sample from a patient and the expression levels of the same one or more selected biomarkers in one or more normal control samples. The expression levels of one or more biomarkers may be greater in the biological sample than in the control sample (i.e., an up-regulated biomarker) or may be less in the biological sample than in the control sample (i.e., a down-regulated biomarker). The threshold for classifying a biomarker expression level as up-regulated may be a constant multiplier of the normal-control-sample expression level, between about 1.05 to about 50, depending on the biomarker and the pathology being diagnosed. Similarly, the threshold for classifying a biomarker expression level as down-regulated may be a constant multiplier of the normal-control-sample expression level, between about 0.9 to about 0.1 or less, depending on the biomarker and pathology being diagnosed.

One embodiment of the present invention comprises a method that distinguishes stage I from stage II and stage III colorectal cancer. The method comprising the following steps: (1) determining the level of expression of one or more biomarkers of Table 1 in a biological sample from a subject having colorectal cancer; (2) comparing the level of the expression of the one or more biomarkers in the biological sample of the subject to the level of expression of the same one or more biomarkers in a normal control or to a predetermined control value to determine the degree of change in expression the one or more biomarkers in the subject sample; (3) comparing the degree of change determined for each biomarker in step (2) to predetermined reference values associated with stage I colorectal cancer. When the degree of change is greater than the predetermined reference value of stage I colorectal cancer for one or more up-regulated biomarkers, and/or the fold change is less than the predetermined reference value of stage I colorectal cancer for one or more down-regulated bio markers, the subject is diagnosed as having stage II or stage III colorectal cancer.

Another embodiment of the invention comprises a method that is used to manage the treatment of colorectal cancer and to monitor the efficacy of colorectal cancer therapy, and to indicate the recurrence of the cancer. The method comprises the following steps: (1) repeatedly determining the level of expression of one or more biomarkers of Table 1 in a biological sample from a subject having colorectal cancer; (2) comparing each of the levels of the expression of the one or more biomarkers in the sample to the levels of expression of the same one or more biomarkers in a normal control group or to a predetermined control value in order to obtain a series of comparisons during the course of a treatment; (3) for each of the one or more biomarkers, when the differences between determined levels of the one or more biomarkers and the corresponding control values decrease, determining that the treatment appears to be effective with respect to the one or more biomarkers; and (4) determining an over-all effectiveness of treatment by determining the ratio of biomarkers with respect to which the treatment appears effective to the total number of biomarkers.

One embodiment of the present invention provides a kit for diagnosing colorectal cancer that can detect the expression of the biomarkers in a biological sample. For example, the kit may include reagants suitable for performing an antibody-based immunoassay, such as an enzyme immunoassay (“ELISA”), a radioimmunoassay (“RIA”), and/or an immunohistochemical test. For example, a kit may comprise binding agents (e.g., antibodies) specific for one or more of the biomarker proteins, or fragments, listed in Table 1. In addition, the kit may comprise one or more of standards, assay diluent, wash buffer, and a solid support such as microtiter plates. In another embodiment of the present invention, a kit may comprise reagents suitable for performing a reverse-transcription polymerase chain reaction (“RT-PCR”) assay that measures nucleic-acid encoding one or more of the protein biomarkers of Table 1. For example, the kit may comprise one or more of a means for isolating total RNA from a biological sample, a means for generating cDNA from isolated total RNA, and pairs of primers suitable for amplifying nucleic acids encoding one or more of the protein biomarkers listed in Table 1.

### Methods for Detecting Biomarkers

Certain method embodiments of the present invention may be practiced by determining the expression level of one or more biomarkers using any technique known in the art. Various techniques may be used to detect mRNA and/or protein levels of biomarkers, including those described below. Mass spectrometry methods are well-known in the art and have been used to quantify and/or identity biomolecules such as proteins. In one embodiment of the present invention, one or more markers listed in Table 1 can be detected and analyzed using chromatographic techniques, such as high pressure liquid chromatograph (“HPLC”) and gel electrophoresis coupled with mass spectrometry, such as tandem mass spectrometry (“MS/MS”), liquid chromatography tandem mass spectrometry (“LC/MS/MS”), matrix assisted laser desorption ionization time-of-flight mass spectrometry (“MALDI-TOF/MS”), and surface enhanced laser desorption ionization mass spectrometry (“SELDI-MS”). One embodiment of the present invention comprises the following steps; (1) determining the level of expression of one or more biomarkers of Table 1 in a biological sample from a subject having, or suspected of having, colorectal cancer; and (2) comparing the concentrations and/or levels of expression of the biomarkers in the biological sample with the corresponding concentrations and/or levels of expression in a normal control group or with predetermined values. In particular embodiments of the present invention, the presence of colorectal cancer is determined when one or more examined biomarkers are differentially expressed above or below up-regulation and down-regulation thresholds, respectively, in the subject sample as compared to a control or are detected at greater or less than threshold concentrations with respect to predetermined values. However, the presence of colorectal cancer may also be determined when the expression level or concentration of one or more of the biomarkers tested is differentially expressed.

In one embodiment of the present invention, biomarkers listed in Table 1 can be identified, analyzed, and quantified using MRM-MS. Specific tryptic peptides can be selected as stoichiometric representatives of the protein markers from which they are cleaved. The selected tryptic peptides can be quantified against a stable isotope-labeled peptide as an internal standard to provide a measure of the concentration of the protein, or can be quantified relatively by comparing the expression level against a normal control. One specific assay comprises a LC/MS/MS based assay coupled with a relative quantitation MRM-MS.

In another embodiment or the present invention, a biomarker is detected in a biological sample by measuring the biomarker protein using an immunoassay, such as Western blotting analysis or an enzyme-linked immunoabsorbent assay (“ELISA”). A variety of immunoassay methods can be used to measure the biomarker proteins. Antibodies specific to the various biomarkers of Table 1 may be readily obtained or produced using standard techniques.

In yet another embodiment, of the present invention, a biomarker in a biological sample is detected by measuring nucleic acid, e.g., mRNA, encoding a protein biomarker of Table 1. In one embodiment of the present invention, the biological sample may be isolated RNA. The detection of RNA transcripts may be achieved by Northern blotting analysis in which a preparation of RNA is run on a denaturing agarose gel and transferred to a suitable support, such as nitrocellulose or nylon membranes. Radiolabeled cDNA or RNA is then hybridized to the preparation, washed, and analyzed by autoradiography. The detection of RNA transcripts may also be achieved by various known amplification methods such as RT-PCR.

### Screening for Therapeutics

Differential expression of biomarkers may be the result of an aberrant expression of the biomarkers at either the genomic (e.g., gene amplification), transcriptomic (e.g., increased mRNA transcription products), or proteomic levels (i.e., translation, post-translational modifications etc.) within a given subject. Aberrant over-expressed biomarkers may be regulated using agents that inhibit their biological activity and/or biological expression, while aberrant under-expressed biomarkers may be regulated using agents that can promote their biological activity or biological expression. Such agents can be used to treat a subject having colorectal cancer, and are referred to as “therapeutic agents”. Agents capable of interacting directly or indirectly with a biomarker in Table 1 can be identified by various methods that are known in the art, such as binding assays, including yeast-2-hybrid and phage display. One embodiment of the present invention provides methods for screening therapeutic agents for treating colorectal cancer resulting from aberrant expression of one or more biomarkers listed in Table 1 below:

TABLE 1
A list of 20 marker proteins that are expressed differently
in a subject having colorectal cancer and a normal control

| | accession no. | Up or | |
|---|---|---|---|
| | swiss-prot | down | |
| Protein Name/Gene Name | Gene ID | regulated | p-value |
| α-1-acid glycoprotein 1 | P02673 | up | 0.042 |
| ORM1 | 5004 | | |
| Gelsolin | P06396 | down | 0.013 |
| GSN | 2934 | | |
| C2 complement | P06681 | up | 0.051 |
| C2 | 717 | | |
| C9 Complement Component | P02748 | up | 0.0082 |
| C9 | 735 | | |
| Pregnancy zone protein | P20742 | up | 0.0094 |
| PZP | 5858 | | |
| C-reactive protein | P02741 | up | 0.0382 |
| CRP | 1401 | | |
| Complement factor H-related | Q03591 | up | 0.0003 |
| protein 1 | 3078 | | |
| CFHR1 | | | |
| Plasma serine protease inhibitor | P05154 | up | 0.0108 |
| SERPINA3 | 5104 | | |
| Hyaluronan-binding protein 2 | Q14520 | up | 0.0111 |
| HABP2 | 3026 | | |
| Beta-Ala-His dipeptidase | Q96KN2 | down | 0.0032 |
| CNDP1 | 84735 | | |
| Complement factor H-related | P36980 | up | 0.0019 |
| protein 2 | 3080 | | |
| CFHR2 | | | |
| Serum amyloid A protein | P02735 | up | 0.0069 |
| SAA | 6288, 6289 | | |
| LOC653879 similar to | P01024 | up | 0.0109 |
| complement C3 | 653879 | | |
| LOC653879 | | | |
| Vitamin K-dependent protein Z | P22891 | down | 0.0029 |
| PROZ | 8858 | | |
| Serum paraoxonase/lactonase 3 | Q15166 | down | 0.0305 |
| PON3 | 5446 | | |
| Retinoic acid receptor responder | Q99969 | up | 0.0023 |
| protein 2 | 5919 | | |
| RARRES2 | | | |
| Gamma-glutamyl hydrolase | Q92820 | up | 0.0041 |
| GGH | 8836 | | |
| proteoglycan-4 | Q92954 | up | 0.0202 |
| PRG4 | 10216 | | |
| Cell surface glycoprotein MUC18 | P43121 | up | 0.006 |
| MCAM | 4162 | | |
| FN1 Isoform 8 of fibronectin | P02751 | up | 0.024 |
| FN1 | 2335 | | |

It should be appreciated that the present invention should not be limited to the biomarkers listed above in Table 1. Additional biomarkers maybe discovered to detect colorectal cancer and other types of cancer of other pathologies.
Appendix 1: provides amino acid sequence for the 20 biomarkers in Table 1.
Appendix 2: provides nucleotide sequence for the 20 biomarkers in Table 1.

### EXAMPLES

The present invention should not be construed to be limited to the examples described here. Embodiments of the present invention include any and all applications provided and all equivalent variations within the skill of the ordinary artisan.

### Example 1

### Identification of Biomarkers for Colorectal Cancer: Discovery Phase

### Test Serum Samples

A total of 14 serum samples were examined, including 8 colorectal cancers: 2 of stage I, 2 of stage II, 2 of stage III, and 2 of stage IV, as well as 6 normal age and gender matched controls.

### Gel-Enhanced LC/MS/MS

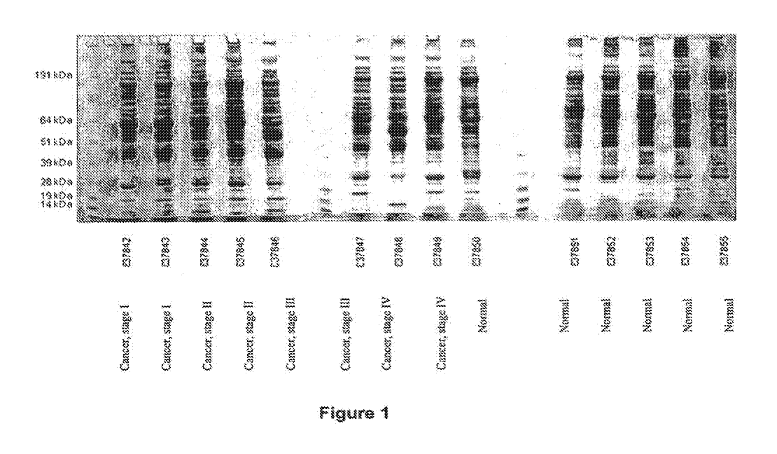

Using the MARS-7 spin column (Agilent), 2 μL of each serum sample was depleted and protein was quantified post-depletion. SDS-PAGE loading buffer was used to solublize 20 μg of each sample. A 4-12% Bis-Tris Novex gel (Invitrogen) was run for each sample in singlet. FIG. 1 shows an SDS-PAGE gel image of 14 serum samples. Each lane 101 to 114 was loaded with 20 μg of each serum sample. The gel was stained with coomassie (SimplyBlue) and then excised into 24 bands per lane using a grid.

### In-Gel Digestion

Each band was subjected to trypsin digestion using a ProGuest workstation as follows: (1) samples were reduced with DTT at 60° C. and allowed to cool to room temperature; (2) samples were alkylated with iodoacetamide and incubated at 37° C. for 4 hours in the presence trypsin; (3) formic acid was added to stop the reaction.

### LC/MS/MS

Gel digests were analyzed using nano LC/MS/MS on a Thermo LTQ Orbitrap XL. 30 μl of hydrolysate were loaded on a 75 μm C12 vented column at a flow-rate of 10 μL/min and eluted at 300 nL/min. A one hour gradient was employed. Product ion data were searched against the IPI Human v3.38 database using the Mascot search engine. The parameters for Mascot searches were as follows:
Type of search: MS/MS Ion Search
Enzyme: Trypsin
Fixed modifications: Carbamidomethyl (C)
Variable modifications: Oxidation (M, Acetyl (N-term, Pyro-glu (N-term Q)
Mass values: Monoisotopic
Protein Mass: Unrestricted
Peptide Mass Tolerance: ±10 ppm (Orbitrap); ±2.0 Da (LTQ)
Fragment Mass Tolerance: ±0.5 Da (LTQ)
Max Missed Cleavages: 1

Mascot output files were parsed into the Scaffold program (www.proteomesoftware.com) for collation into non-redundant lists per lane and filtering to assess false discovery rates and allow only correct protein identifications. Spectral counts per protein were output. These spectral counts constitute a semi-quantitative measure of abundance across samples. Spectral count reflects the number of matched peptides and the number of times those peptides were observed

### RESULT

A total of 435 proteins were identified. Using the boxplot and student t-test methods, 42 proteins were identified having a p-value<0.05. Further statistical analysis was performed using exploratory data analysis and principal component analysis with S-plus statistical software to examine the differentiation between diseased samples and normal samples of the 42 markers. Results from the principal component analysis along with biological/clinical relevance of the markers led to the set of 20 biomarkers listed in Table 1. MRM-MS assay was set-up to validate those 20 proteins as described in Example 2. Based on the quality of the fragmentation data of the product ion spectra, 10 proteins were chosen

### Example 2

### Immunodetection of the Biomarkers

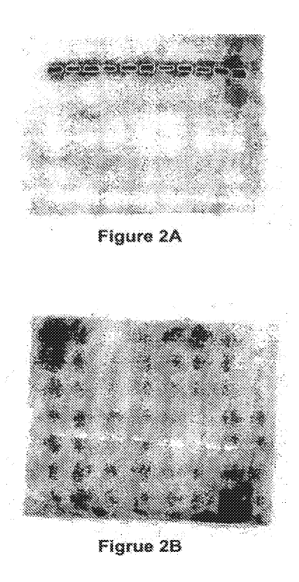

To validate the expression of the biomarkers listed in Table 1, western immunoblotting was performed. Antibodies for ORM1, GSN, SAA, PROZ, PON3, MCAM1, PZP, and FN1 were obtained from Santa Cruz Biotechnology Inc. A 1 μl aliquot of each of 11 serum samples from the discovery phase was solublized in SDS-PAGE loading buffer and loaded onto a 4-12% Bis-Tris Novex gel (Invitrogen). The gel was electrophoreses at 120 volts for 60 minutes and the serum proteins separated in the SDS-PAGE gel were transferred to a nitrocellulose membrane using Bio-Rad semi-dry electroblotting unit for 90 min. The blot was blocked with Starting Block™ Blocking Buffer (Pierce) and incubated overnight at 4° C. with primary antibody followed by three 10-minute washes with TBS containing 0.05% Tween 20 (TBS-T). The blot was then incubated with a Horseradish Peroxidase (“HRP”) conjugated secondary antibody for one hour at room temperature and then washed four times in TBS-T for 15 minutes each time. Signal detection was achieved using SuperSignal Substrate (Pierce) and the blots were imaged using the Kodak 2000 Image Station. FIGS. 2A and 2B show western blots for colorectal cancer and normal serum samples probed with 1 μg/ml rabbit polyclonal anti human fibronectin antibodies and 1 μg/ml of rabbit IgG1 isotype antibodies as a negative control. FIG. 2A shows a western blot for colorectal cancer and normal serum samples probed with 1 μg/ml rabbit polyclonal anti human fibronectin antibodies, and FIG. 2B shows a western blot for colorectal cancer and normal serum samples probed with rabbit IgG1 isotype antibodies as a negative control. From left to right in both FIG. 2A and FIG. 2B: lane 1: stage I colon cancer sample, lane 2: normal, age and gender matched with colon cancer samples in lane 1 and lane 3; lane 3: stage IIA colon cancer sample; lane 4: normal, age and gender matched with lane 5; lane 5: stage IIIA colon cancer sample; lane 6: stage IIIB colon cancer sample; lane 7: normal, age and gender matched with colon cancer sample in lane 6; lane 8: stage IV colon cancer sample; lane 9: normal, age and gender matched with colon cancer sample in lane 8; lane 10: stage I colon cancer sample; land 11: normal, age and gender matched with colon cancer sample in lane 10. Each lane was loaded with 1 μl of serum sample.

### Example 3

### Development and Validation of an MRM-MS Assay MRM-MS Peptide Panel Selection

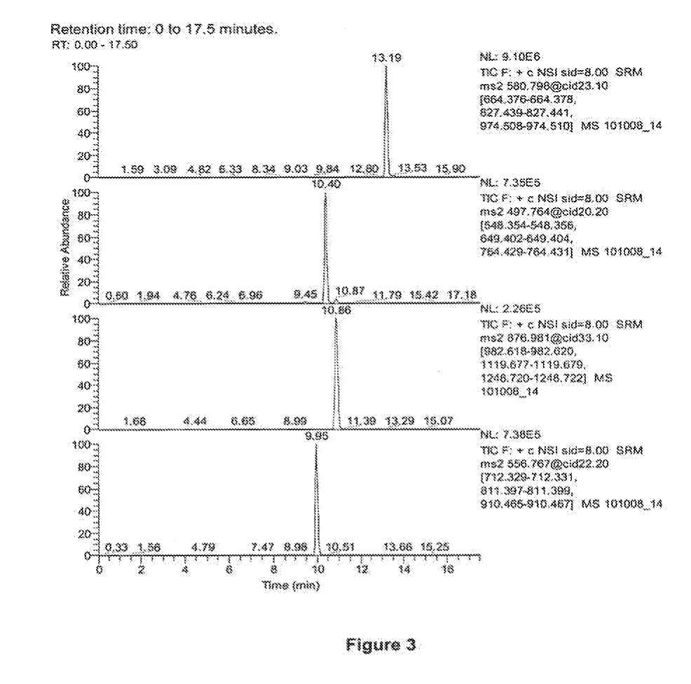

Peptides of the biomarkers listed in Table 1 were selected from discovery data for MRM-MS assay development. All proteins were tested individually in panels with peptides amenable to MRM-MS-assay analysis on pooled disease and control samples. For peptides that worked the best, the method was reduced to a scan for the two most abundant product ions, and the method was run again against both the disease and control pool again. The 10 proteins were chosen as an MRM-MS panel for multiplexing with the best peptide(s) selection per protein. FIG. 3 shows representative selected-reaction-monitoring mass spectrometry (“SRM-MS”) chromatograms for α-1-acid glycoprotein 1 (“ORM 1”) working peptides.

### 10-Plex Relative Protein MRM-MS Assay

A 10-plex relative protein assay was developed for these 10 biomarkers. A summary of the sequences of the transition peptides used for MRM-MS assay for the 10 biomarkers is listed in Table 3. The performance of the assay was determined by analyzing normal samples in Example 1 in triplicate from the sample preparation through mass spectrometry to evaluate the reproducibility of the assay.

### Depletion

(1) 15 μL of serum was depleted using the MARS7 spin column (Agilent) according to the manufacturers protocol; (2) samples were buffer exchanged using a 5 KDa MWCO spin filter into 25 mM ammonium bicarbonate; (3) a Bradford protein quantitation assay was performed.

### Solution Digestion

Samples were subjected to proteolytic digestion as follows: (1) reducing with DTT at 60° C. and allowed to cool to room temperature; (2) alkylating with iodoacetamide and incubated at 37° C. for 18 h in the presence of trypsin; and (3) adding formic acid to stop the reaction, followed by direct analysis of the supernatant.

### LC/MRM-MS

Peptides were separated using a 15 cm×100 μm ID column packed with a 4 μm C12 resin (Jupiter Proteo, Phenomenex) under gradient conditions at a constant flow rate of 800 nL/min. The gradient is outlined in Table 2. The composition of solvent A was water containing 0.1% formic acid and 0.1% acetonitrile and the composition of solvent B was acetonitrile containing 0.1% formic acid. Samples were loaded onto the column using a trapping strategy. An injection volume of 30 μL was used and the experiment was optimized so that 500 ng of peptide was loaded on the column per sample. The total runtime, injection to injection, was 20 minutes.

TABLE 2
Outline of LC gradient for Solvent A and Solvent B

| Time (min) | % Solvent A | % Solvent B |
|---|---|---|
| 0.00 | 99 | 1 |
| 10.00 | 75 | 25 |
| 12.00 | 50 | 50 |
| 13.00 | 5 | 95 |
| 14.00 | 5 | 95 |
| 14.10 | 99 | 1 |
| 17.00 | 99 | 1 |

A ThermoFinnigan tandem quadrupole (“TSQ Ultra”) mass spectrometer was used for peptide detection in SRM mode. Mass spectrometer settings included a spray voltage of 2.2 kV and capillary temperature of 250° C. A 0.2 FWHM resolution in Q1 (hSRM) and 0.7 FWHM resolution in Q3 were employed. Argon was used as a collision gas at a pressure of 1.5 mTorr. The dwell time for each SRM transition was 10 ms. All MRM-MS experiments were conducted in triplicate and the data were processed using the LCQuan software package (ThermoFinnigan).

### RESULT

A summary of the percent analytical relative standard deviation (“% RSD”) and technical % RSD of one transition peptide for all 10 proteins is given in Table 4. Low analytical % RSD (<10%) and technical % RSD (<20%) for transition peptides were achieved, except for the C2 protein. Analytical % RSD is the percent relative standard deviation from a single sample processed via 3 injections; technical % RSD is the percent relative standard deviation from one sample processed 3 times (3 depletion, 3 digestions) with one injection for each 3 separated run.

TABLE 3
Summary of transition peptides for 10 proteins

| | Transition peptides sequences | | |
|---|---|---|---|
| Proteins | Peptide 1 | Peptide 2 | Peptide 3 |
| ORM1 | SEQ ID NO 1: | SEQ ID NO 2: | SEQ ID NO 3: |
| | WFYIASAFG | TEDTIFLR | SDWYTDWK |
| GSN | SEQ ID NO 4: | SEQ ID NO 5: | SEQ ID NO 6: |
| | IFVWK | QTQVSVLPEGGETP | AGALNSNDAFVL |
| | | LFK | K |
| C9 | SEQ ID NO 7: | SEQ ID NO 8: | SEQ ID NO 9: |
| | YAFELK | LSPIYNLVPVK | AIEDYIEFSVR |
| FN1 | SEQ ID NO 10: | SEQ ID NO 11: | SEQ ID NO 12: |
| | WLPSSSPVTGYR | IYLYTLNDNAR | SYTITGLQPGTD |
| | | | YK |
| SERPIN | SEQ ID NO 13: | SEQ ID NO:14: | SEQ ID NO 15: |
| A3 | EQLSLLDR | EIGELYLPK | ITLLSALVETR |
| PZP | SEQ ID NO 16: | SEQ ID NO 17: | |
| | ATVLNYLPK | AVGYLITGYQR | |
| C2 | SEQ ID NO 18: | SEQ ID NO 19: | |
| | HAFILQDTK | AVISPGFDVAK | |
| PROZ | SEQ ID NO 20: | | |
| | GLLSGWAR | | |
| PRG4 | SEQ ID NO 21 | | |
| | AIGPSQTHTIR | | |
| SAA2 | SEQ ID NO 22: | | |
| | SFFSFLGEAFDG | | |
| | AR | | |

TABLE 4
Summary of analytical and technical % RSD
of one transition peptide for 10 proteins

Peptide 1

| | Analytical | Technical |
|---|---|---|
| Proteins | % RSD | % RSD |
| ORM1 | 3% | 8% |
| GSN | 9% | 15% |
| C9 | 7% | 12% |
| FN1 | 1% | 17% |
| SERPINA3 | 6% | 11% |
| PZP | 5% | 18% |
| C2 | 16% | 25% |
| PROZ | 8% | 12% |
| PRG4 | 3% | 11% |
| SAA2 | 10% | 24% |

The selected tryptic peptides can be quantified against a stable isotope-labeled peptide as an internal standard to provide a measure of the concentration of the protein, or can be quantified relatively by comparing the expression level against a normal control

### Example 4

### Validation of 10 Biomarkers with the First Expanded 33 Serum Sample Set

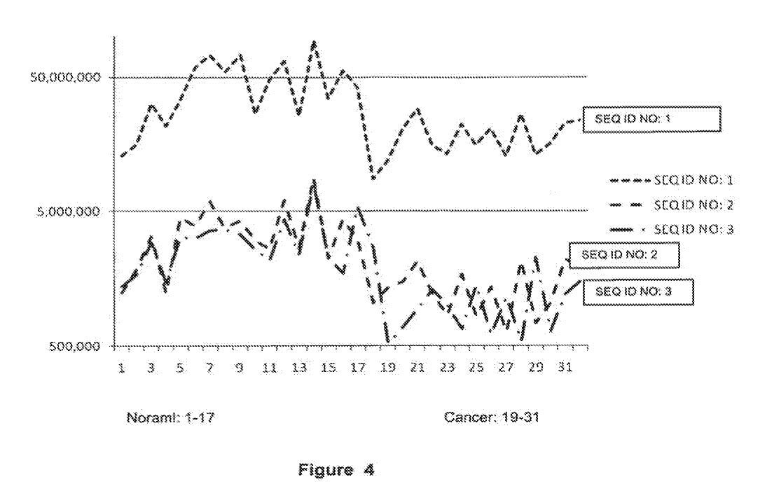

The relative level of 10 protein biomarkers of colon cancer was monitored in 33 patient serum samples as well as in the 13 samples used for biomarker discovery. This larger sample set included 18 age-and-gender-matched normal serum samples and 15 colon cancer serum samples: four Stage I, five Stage II, eight Stage III and one Stage IV. The 33 serum samples were collected from the same institute using the same collection protocol. Thirteen of the fourteen original samples from Example 1 were also tested in the assay. All samples were processed with equivalent amounts of protein. Data from two analytical replicates of each sample were collected and analyzed. The data summary below reports the ratio of the average data for each protein across the samples from a particular stage of disease relative to the average for the normal group. FIG. 4 shows representative MRM-MS peptide trend lines of three ORM1 working peptides for 33 serum samples. Sample ID numbers from 38715 to 38732 are normal subjects; sample ID numbers from 38733 to 38750 are from subjects with colon cancer.

### Sample Preparation Steps

The sample order was randomized so that samples from the same group were not processed in sequence. Samples were prepared following the steps: (1) depletion; (2) solution digestion, as described in Example 3, except that the samples were placed in a 96 well plate and digested with trypsin overnight.

### LC/MRM-MS

An internal standard was added to each sample. Samples were then tested following the same LC/MRM-MS condition as described in Example 3.

### RESULT

| | Stage 1: | | Stage 2: | | Stage 3: | | Stage 4: | |
|---|---|---|---|---|---|---|---|---|
| sample | Normal | p-val | Normal | p-val | Normal | p-val | Normal | p-val |
| SERPINA3 | 2.04 | 0.008151 | 3.74 | 0.000243 | 4.60 | 0.011396 | 1.79 | NA |
| FN1 | 0.71 | 0.347941 | 0.59 | 0.125433 | 0.79 | 0.460251 | 0.45 | NA |
| SAA2 | 6.43 | 0.006389 | 19.88 | 0.001009 | 14.24 | 0.012542 | 31.60 | NA |
| PROZ | 0.80 | 0.331598 | 0.69 | 0.186481 | 1.08 | 0.701716 | 0.61 | NA |
| PZP | 0.91 | 0.835463 | 1.73 | 0.088589 | 2.20 | 0.030897 | 0.85 | NA |
| C9 | 1.61 | 0.026879 | 2.73 | 0.000015 | 2.91 | 0.000366 | 2.55 | NA |
| PRG4 | 1.04 | 0.919156 | 1.65 | 0.182810 | 1.78 | 0.040367 | 1.34 | NA |
| C2 | 1.12 | 0.805110 | 2.16 | 0.006040 | 1.55 | 0.130820 | 0.87 | NA |
| GSN | 0.55 | 0.022829 | 0.30 | 0.000224 | 0.83 | 0.447100 | 0.28 | NA |
| ORM1 | 1.41 | 0.095748 | 3.24 | 0.000000 | 3.24 | 0.000054 | 2.37 | NA |

All ten of the biomarkers showed differential expression. Seven of the ten biomarkers discovered were confirmed in this set of serum samples as differentially expressed between the cancer and normal groups with a p-value less than 0.05 in one or more stages of colorectal cancer.

### Statistical Analysis

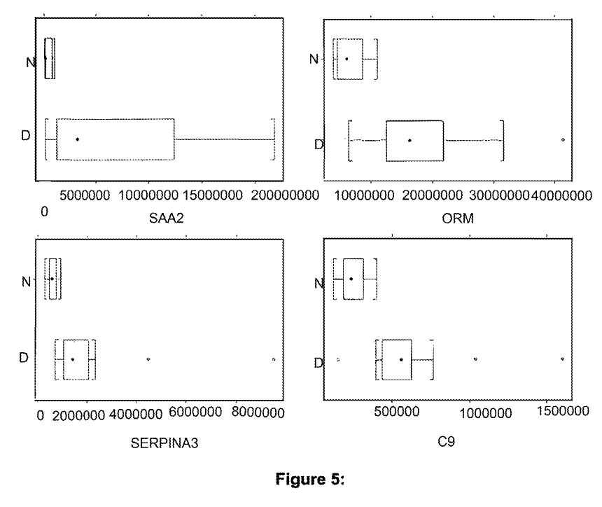

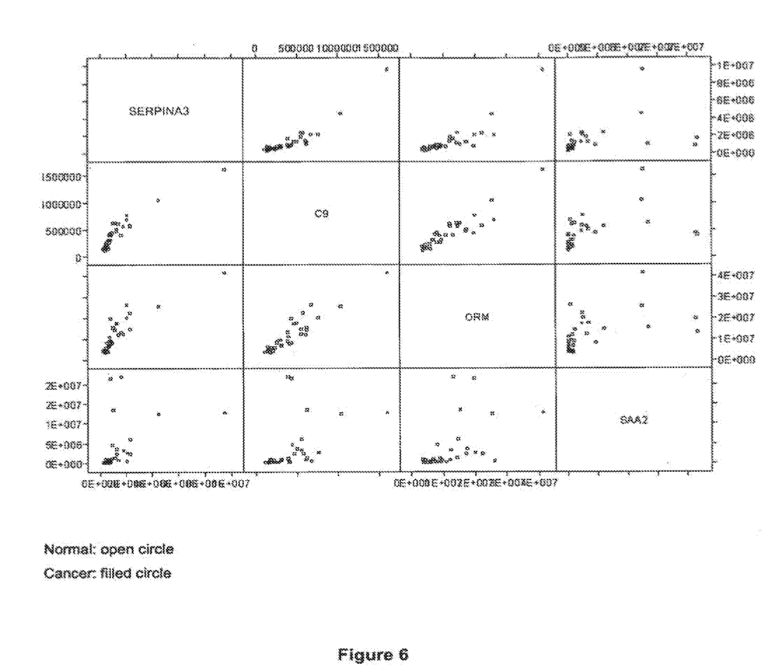

Using S-plus statistical software, exploratory data analysis, multivariate analysis, and discriminant analysis were performed. FIG. 5 shows boxplots of cancer vs. normal for serum values tor amyloid A protein (“SAA2”), ORM1, plasma serine protease inhibitor (“SERPINA3”), and C9 complement component (“C9”). In FIG. 5, the x-axis indicates the spectral counts and the y-axis indicates the sample category: Cancer and Normal. FIG. 6 shows a matrix plot for SERPINA3, ORM1, SAA2, and C9. The labels along the x and y axes indicates the spectral counts. The circle indicates the normal controls, and the triangles are the cancer group. A separation between cancer and normal control is observed, which suggests that the difference between the cancer and normal states may be related to a combination of these variables (i.e., biomarkers).

Multivariate analysis and discriminant analysis was carried out for different combinations of multiple biomarkers using 4 markers as classifiers of diseased state vs. normal state: C9, ORM1, SAA2, and SERPINA3. 16 of the 17 cancer samples were classified correctly as cancer, and 16 of the 15 normal samples were classified as normal The plug-in classification table, using 4 markers shown below, is the output result from S-plus software:

| | Diseased | Normal | Error | Posterior Error |
|---|---|---|---|---|
| Diseased | 16 | 1 | 0.0588235 | 0.00695156 |
| Normal | 0 | 15 | 0.0000000 | 0.0653836 |
| Overall | | | 0.0588235 | 0.0062816 |

Discriminant analysis used 7 markers as classifiers of diseased state vs. normal state: C9, ORM1, SAA2, SERPINA3, PZP, PRG4, and PROZ. 16 of the 17 cancer samples were classified correctly as cancer, and 15 of the 15 normal samples were classified as normal. Output result from S-plus software is shown below in classification table using 7 markers:

| | Diseased | Normal | Error | Posterior Error |
|---|---|---|---|---|
| Diseased | 16 | 1 | 0.0588235 | 0.0588235 |
| Normal | 0 | 15 | 0.0000000 | 0.0663935 |
| Overall | | | 0.0588235 | 0.0001280 |

Discriminant analysis using 8 markers: C9, ORM1, SAA2, SERPINA3, PZP, PRG4, PROZ, and GSN as classifiers of diseased state vs. normal state with heteroscedastic covariance structure. 17 of the 17 cancer samples were classified correctly as cancer, and 15 of the 15 normal samples were classified as normal. Output result from S-plus software as plug-in classification table is shown below:

| | Diseased | Normal | Error | Posterior Error |
|---|---|---|---|---|
| Diseased | 17 | 0 | 0 | 0.0e+000 |
| Normal | 0 | 15 | 0 | 9.8e−006 |
| Overall | | | 0 | 4.6e−006 |

The use of multiple biomarkers increases the predictive value of the test monitoring.

### Example 5

### Validation of 10 Biomarkers in Plasma Sample Set

The 10-plex relative protein MRM-MS assay was tested in plasma samples. All samples were collected before treatment and before surgery. 5 plasma colorectal cancer samples, including one Stage I, two Stage II, two Stage III, and one pooled normal plasma sample were tested along with a normal serum and a colorectal cancer serum samples. Samples were prepared and processed to LC/MRM-MS as described in Example 3.

### LC/MRM-MS

An internal standard was added to each sample. Samples were then tested following the same LC/MRM-MS condition, as described in Example 3.

### RESULT

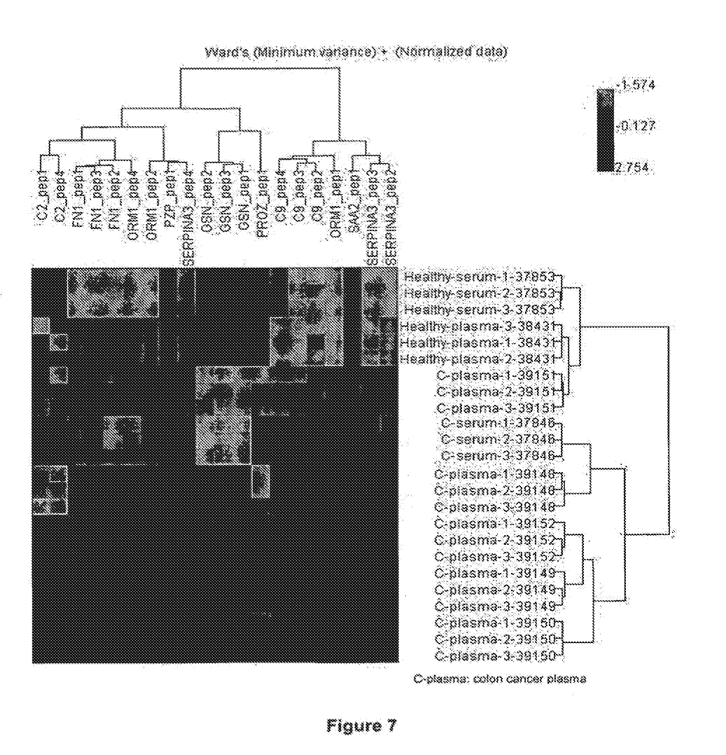

Other than PROG4, all biomarkers were detected. All of the transition peptides of the nine biomarkers discovered were confirmed, except PZP_pep2 (SEQ ID NO: 17) in this set of plasma samples as differential expressed between the cancer and normal groups. The degree of change and the p-value for each transition peptide of the 9 biomarkers are listed in Table 5. All the transition peptides of the nine biomarkers showed a p-value less than 0.05, except C2_pep1 (SEQ ID NO: 18), SAA2 (SEQ ID NO: 22), and PROZ_pep1 (SEQ ID NO 20). FIG. 7 shows a hierarchical clustering analysis of plasma and serum samples. FIG. 7 is the Hierarchical Clustering Analysis of plasma and serum samples, where C-plasma denoted Colon cancer plasma sample.

TABLE 5
Fold change and p-value of the transition peptides for the 9
biomarkers detected

| | Transition | Fold change | |
|---|---|---|---|
| Proteins | peptides | cancer: | |
| peptids | sequences | normal | P-value |
| C2_pep1 | SEQ ID NO 18: | 6.95 | 0.217062141 |
| | HAFILQDTK | | |
| C2_pep2 | SEQ ID NO 19: | 2.78 | 0.002870429 |
| | AVISPGFDVFAK | | |
| C9_pep1 | SEQ ID NO 7: | 2.83 | 1.30572E-05 |
| | YAFELK | | |
| C9_pep2 | SEQ ID NO 8: | 4.49 | 1.05609E.06 |
| | LSPIYNLVPVK | | |
| C9_pep3 | SEQ ID NO 9: | 4.50 | 0.000124535 |
| | AIEDYIEFSVR | | |
| FN1_pep1 | SEQ ID NO 10: | 3.34 | 0.000265144 |
| | WLPSSSPVTGYR | | |
| FN1_pep2 | SEQ ID NO 11: | 3.22 | 0.000460216 |
| | IYLYTLNDNAR | | |
| FN1_pep3 | SEQ ID NO 12: | 3.39 | 0.000148397 |
| | SYTITGLQPGTDYK | | |
| GSN_pep1 | SEQ ID NO 4: | 1.71 | 0.021196134 |
| | IFVWK | | |
| GSN_pep2 | SEQ ID NO 5: | 2.46 | 0.00175376 |
| | QTQVSVLPEGGETPLF | | |
| | K | | |
| FN1_pep3 | SEQ ID NO 6: | 1.63 | 0.041756746 |
| | AGALNSNDAFVLK | | |
| ORM1_pep1 | SEQ ID NO 1: | 5.85 | 2.14067E-05 |
| | WFYIASARF | | |
| ORM1_pep2 | SEQ ID NO 2: | 4.13 | 0.000362804 |
| | TEDTIFLR | | |
| ORM1_pep3 | SEQ ID NO 3: | 3.31 | 9.58596E-06 |
| | SDWYTDWK | | |
| SAA2_pep1 | SEQ ID NO 22: | 1750.2 | 0.258875784 |
| | SFFSFLGEAFDGAR | | |
| PZP_pep1 | SEQ ID NO 16: | 2.09 | 0.090043107 |
| | ATVLNYLPK | | |
| PROZ_pep1 | SEQ ID NO 20: | 7.37 | 0.346022569 |
| | GLLSGWAR | | |
| SERPINA3_ | SEQ ID NO 13: | 3.72 | 7.20014E-05 |
| pep1 | EQLSLLDR | | |
| SERPINA3_ | SEQ ID NO 14: | 4.87 | 0.000202835 |
| pep2 | EIGELYLPK | | |
| SERPINA3_ | SEQ ID NO 15: | 4.27 | 0.001260361 |
| pep3 | ITLLSALVETR | | |

### Example 6

### Validation of 10 Biomarkers with 2^{nd} Expended 48 Serum Samples

The relative levels of the 10 protein biomarkers of colon cancer was further confirmed and validated by obtaining relative quantitation data from 48 serum samples, including samples from healthy individuals and patients with colon cancer. The 48 serum samples were collected from the same institution as the 33 serum samples in Example 4. Of the 48 samples, 24 were from healthy individuals confirmed by negative colonoscopy and 24 from colorectal cancer patients with different stages, including one from Stage I, twelve from Stage II, six from Stage III, and five from Stage IV.

### Sample Preparation Steps

The sample order was randomized so that samples from the same group were not processed in sequence. Each sample was processed in analytical triplicate (same processed sample on Mass spectrometry three times). Samples were prepared following the steps: (1) depletion; and (2) solution digestion, as described in Example 3, except that the samples were placed in a 98-well plate and digested with trypsin overnight.

### LC/MRM-MS

An internal standard was added to each sample. Samples were then tested following the same LC/MRM-MS condition, as described in Example 3. FIG. 3 shows an MRM-MS C9 trend line for three transition peptides of the C9 protein.

### RESULT

Eight of the 10 proteins were detected in these samples, PRG4 and SAA2 were not detected. Four of the proteins (10 peptides) had p values less than 0.05 comparing the healthy group to the group with colon cancer. The 4 proteins are: C9, FN1, GSN and SERPINA3. Table 6 provides the p values (t-Test) for the different groups (all cancer stages, Stage II, Stage III, and Stage IV compared to the healthy group). Because only one Stage I sample was included in the testing, there is no p value for that sample.

TABLE 6
Results at the protein level: p-values of 8 proteins detected from
for each group compared to control group

| | C2 | C9 | FN1 | GSN | ORM1 | PROZ | PZP | SERPINA3 |
|---|---|---|---|---|---|---|---|---|
| Stage II | 0.797 | 0.054 | 0.001 | 0.264 | 0.682 | 0.241 | 0.657 | 0.121 |
| Stage III | 0.455 | 0.006 | 0.004 | 0.029 | 0.242 | 0.757 | 0.443 | 0.041 |
| Stage IV | 0.694 | 0.409 | 0.529 | 0.170 | 0.283 | 0.279 | 0.556 | 0.006 |
| All cancer | 0.825 | 0.020 | 0.001 | 0.022 | 0.493 | 0.891 | 0.526 | 0.016 |

TABLE 7
Results at peptides level: fold change and p-value of the
transition peptides for the 8 biomarkers detected

| | Transition | Fold change | |
|---|---|---|---|
| Proteins | peptides | cancer: | |
| peptids | sequences | normal | P-value |
| C2_pep1 | SEQ ID NO 18: | 1.06 | 0.54551 |
| | HAFILQDTK | | |
| C2_pep2 | SEQ ID NO 19: | 1.07 | 0.489746 |
| | AVISPGFDVFAK | | |
| C9_pep1 | SEQ ID NO 7: | 1.65 | 0.000325 |
| | YAFELK | | |
| C9_pep2 | SEQ ID NO 8: | 1.52 | 0.007926 |
| | LSPIYNLVPVK | | |
| C9_pep3 | SEQ ID NO 9: | 1.65 | 0.010036 |
| | AIEDYIEFSVR | | |
| FN1_pep1 | SEQ ID NO 10: | 0.72 | 0.008734 |
| | WLPSSSPVTGYR | | |
| FN1_pep2 | SEQ ID NO 11: | 0.67 | 0.000948 |
| | IYLYTLNDNAR | | |
| GSN_pep1 | SEQ ID NO 4: | 0.80 | 0.001601 |
| | IFVWK | | |
| GSN_pep2 | SEQ ID NO 5: | 0.80 | 0.140187 |
| | QTQVSVLPEGGETPLF | | |
| | K | | |
| FN1_pep3 | SEQ ID NO 6: | 0.81 | 0.009414 |
| | AGALNSNDAFVLK | | |
| ORM1_pep1 | SEQ ID NO 1: | 1.31 | 0.190992 |
| | WFYIASARF | | |
| ORM1_pep2 | SEQ ID NO 2: | 1.10 | 0.493723 |
| | TEDTIFLR | | |
| ORM1_pep3 | SEQ ID NO 3: | 1.22 | 0.222723 |
| | SDWYTDWK | | |
| PROZ_pep1 | SEQ ID NO 20: | 1.00 | 0.982778 |
| | GLLSGWAR | | |
| PZP_pep1 | SEQ ID NO 16: | 1.10 | 0.212562 |
| | ATVLNYLPK | | |
| PZP_pep2 | SEQ ID NO 17: | 1.16 | 0.674164 |
| | AVGYLITGYQR | | |
| SERPINA3_ | SEQ ID NO 13: | 1.60 | 0.002006 |
| pep1 | EQLSLLDR | | |
| SERPINA3_ | SEQ ID NO 14: | 1.50 | 0.006981 |
| pep2 | EIGELYLPK | | |
| SERPINA3_ | SEQ ID NO 15: | 1.58 | 0.005854 |
| pep3 | ITLLSALVETR | | |

### Statistical Analysis

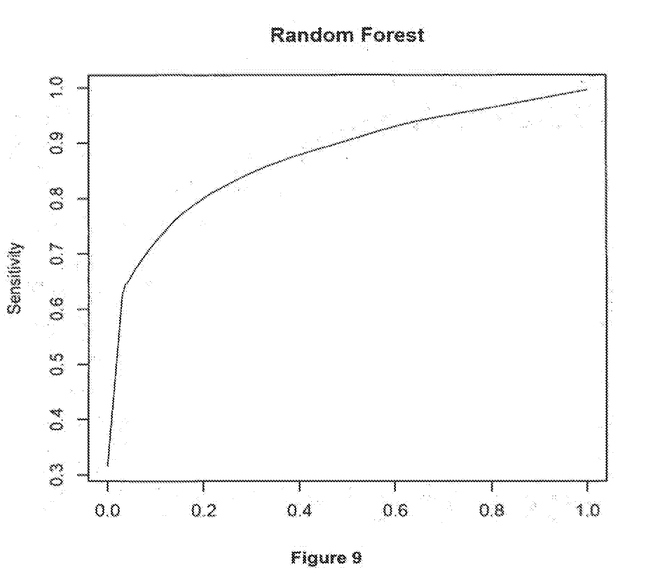

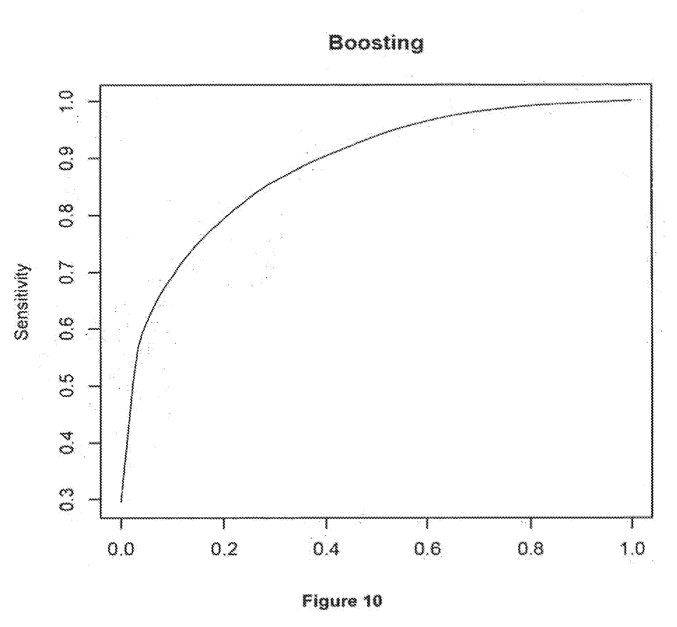

A total of 17 peptides from Table 5, with C2_pep1 (SEQ ID NO 18), PROZ_pep1 (SEQ ID NO 20) and PZP_pep2 (SEQ ID NO 17) excluded, were used for statistical analysis. Two classification methods, Random Forest and Boosting, were used to construct Receiver operating Characteristic (“ROC”) curves to assess the diagnostic accuracy of the biomarkers in distinguishing patients with colon cancer from control subjects. The analysis was performed as follows: (1) 48 serum samples were randomly split into a training set of 32 samples and a test set of 16 samples; (2) with a given random split, the training set data was fitted into Random Forest and Boosting models. They were evaluated on the test data set, and variable importance and area under ROC curves were recorded; and (3) steps (1) and (2) were repeated 100 times based on 100 random splits. The final results were averaged over the 100 random splits. FIG. 9 shows a Receiver Operating Characteristic (“ROC”) curve for a 48-serum-sample set using the random-forest model. FIG. 9 is the Receiver Operating Characteristic (“ROC”) curve for 48 serum samples set using Random Forest model. The area under curve (“AUC”) was 0.868 with an 8.7% standard deviation. FIG. 10 shows an ROC curve for 48 serum samples using the boosting method. The area under curve (“AUC”) is 0.901 and a standard deviation of 5.4% was obtained. Based on both methods, a sensitivity of 80% and specificity of 80% were obtained.

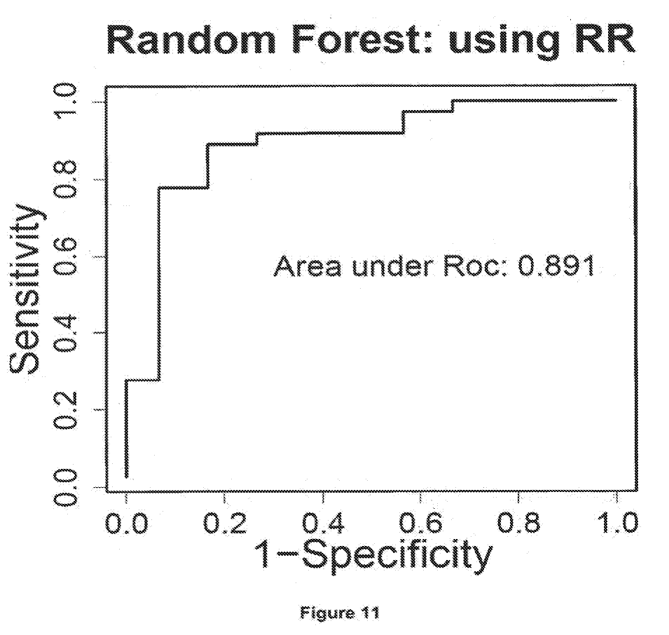

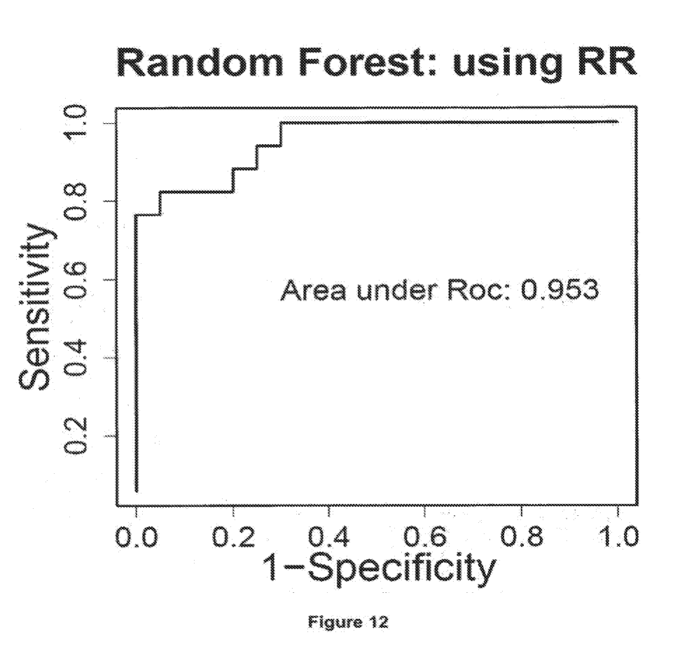

FIG. 11 shows an ROC curve for a 33-serum-sample set constructed by the random-forest model. Further statistical analysis based on the Random Forest model was performed using 48 samples set as training set to test the 33 serum samples set in Example 4. The ROC curve is shown in FIG. 11 with an AUC of 0.891. A specificity of 90% and sensitivity of 85% was drawn based on the ROC curve. Statistical analysis using Random Forest method was also carried out for a set of 33 serum samples and for the set of 13 serum samples in Example 4. FIG. 12 shows an ROC curve for a 13-serum-sample set constructed by the random-forest model. The ROC curve is shown in FIG. 12 using a set of 33 serum samples as a training set to test the 13 serum samples set An AUC of 0.953 was obtained and giving a sensitivity of around 83% and specificity around 95%.

### Example 7

### Absolute Quantitative Assay Development

An absolute quantitative MRM-MS assay using stable isotope dilution mass spectrometry was further developed. In this study, quantification of proteins is accomplished by selecting “signature” peptides derived by trypsin digestion of the target protein released during sample digestion. These signature peptides, unique in sequence (i.e. not present in other proteins in the genome), are used as quantitative, stoichiometric surrogates of the protein itself. When a synthetic, stable isotope-labeled version is used as an internal standard, protein concentration can be measured by comparing the signals from the exogenous labeled and endogenous unlabeled species. To insure optimal performance of peptide standards used for quantitation, alternative peptides other than the transition peptides listed in Table 3 for each protein in Table 1 were also evaluated.

### Peptide Selection and Detection:

Peptides candidates for each protein were generated based on their presence in the serum spectral library, which is a collation of all peptides observed during discovery experiments described in Example 1, and their physical properties, such as size, amino acid composition.

The six control and six disease samples were used to generate two pools: pool-control and pool-disease. A method for each protein including all the peptides candidates was used to run both samples. The mass chromatograms were inspected visually using the Skyline program. Peptides were eliminated when they fell into one or more of these criteria: (1) no peak in either sample; (2) peak defected, but product ion ratio is not similar; (3) multiple peaks detected which could cause potential interference. A variance test was also performed where a pooled sample (3 controls and 3 diseases) was prepared in triplicate (varA, varB, and varC). Each of these three samples was then analyzed in triplicate. Samples were processed following the steps: (1) depletion; and (2) solution digestion, as described in Example 3, except that 10 μl of serum was depleted instead of 15 μl of serum. Samples were then tested following the same LC/MRM-MS condition as described in Example 3. Peptides with analytical and technical variance greater than 20% were eliminated.

### Multiplex Assay Testing:

Based on the peptides selected, a multiplex assay was constructed. To further evaluate the robustness of the multiplex assay with the selected signature peptides, a variance test and a pilot test were carried out. Table 8 is a list of selected peptides for the multiplex assay and pilot testing.

TABLE 8

| Proteins | Selected signature peptides sequences | | |
|---|---|---|---|
| ORM1 | SEQ ID NO 1: | SEQ ID NO 2: | SEQ ID NO 3: |
| | WFYIASAFG | TEDTIFLR | SDWYTDWK |
| | SEQ ID NO 23 | | |
| | YVGGQEHFAHLLIL | | |
| | R | | |
| GSN | SEQ ID NO 4: | SEQ ID NO 5: | SEQ ID NO 6: |
| | IFVWK | QTQVSVLPEGGETPL | AGALNSNDAFVL |
| | | FK | K |
| | SEQ ID NO 24 | SEQ ID NO 25 | |
| | HVVPNEVVVQR | SEDCFILDHGK | |
| C9 | SEQ ID NO 8: | SEQ ID NO 9: | SEQ ID NO 26 |
| | LSPIYNLVPVK | AIEDYIEFSVR | SIEVFGQFNGK |
| | SEQ ID NO 27 | | |
| | TSNFNAAISLK | | |
| FN1 | SEQ ID NO 10: | SEQ ID NO 11: | SEQ ID NO 12: |
| | WLPSSSPVTGYR | IYLYTLNDNAR | SYTITGLQPGTD |
| | | | YK |
| | SEQ ID NO 28 | | |
| | VTWAPPPSIDLTNF | | |
| | LVR | | |
| SERPINA3 | SEQ ID NO 13: | SEQ ID NO 15: | SEQ ID NO 29 |
| | EQLSLLDR | ITLLSALVETR | ADLSGITGAR |
| | SEQ ID NO 30: | | |
| | AVLDVFEEGTEASA | | |
| | ATAVK | | |
| PZP | SEQ ID NO 16: | SEQ ID NO 17: | SEQ ID NO 31: |
| | ATVLNYLPK | AVGYLITGYQR | SLFTDLVAEK |
| | SEQ ID NO 32: | SEQ ID NO 33: | |
| | NQGNTWLTAFVLK | SSGSLLNNAIK | |
| C2 | SEQ ID NO 18: | SEQ ID NO 19: | SEQ ID NO 34: |
| | HAFILQDTK | AVISPGFDVFAK | ECQGNGVWSG |
| | | | TEPICR |
| | SEQ ID NO 35 | SEQ ID NO 36: | |
| | EILNINQK | DFHINLFR | |
| PROX | SEQ ID NO 24: | SEQ ID NO 37: | SEQ ID NO 38: |
| | GLLSGWAR | APDLQDLPWQVK | ENFVLTTAK |
| | SEQ ID NO 39: | | |
| | YSLWFK | | |
| PRG4 | SEQ ID NO 40: | SEQ ID NO 41: | SEQ ID NO 42: |
| | ITEVWGIPSPIDTVF | GFGGLTGQIVAALST | IQYSPAR |
| | TR | AK | |
| | SEQ ID NO 43: | SEQ ID NO 44: | |
| | DQYYNIDVPSR | CFESFER | |
| SAA2 | SEQ ID NO 22: | SEQ ID NO 45: | SEQ ID NO 46: |
| | SFFSFLGEAFDGAR | GPGGWVAAEAISDAR | FFGHGAEDSLA |
| | | | DQAANEWGR |
| CFHR2 | SEQ ID NO 47: | SEQ ID NO 48: | SEQ ID NO 49: |
| | ITCAEEGWSPTPK | GWSTPPK | TGDIVEFVCK |
| | SEQ ID NO 50: | | |
| | LVYPSCEEK | | |
| LOC653879 | SEQ ID NO 51: | SEQ ID NO 52: | SEQ ID NO 53: |
| | IHWESASLLR | NTLIIYLDK | VYAYYNLEESCT |
| | | | R |
| | SEQ ID NO 54: | SEQ ID NO 55: | |
| | ACEPGVDYVYK | TFISPIK | |
| HABP2 | SEQ ID NO 56: | SEQ ID NO 57: | SEQ ID NO 58: |
| | FTCACPDQFK | VVLGDQDLK | LIANTLCNSR |
| | SEQ ID NO 59: | | |
| | FLNWIK | | |

### Variance Test

A pooled sample comprising three controls and three diseases was prepared three times to give the following samples: varA, varB, and varC. Each of these three samples was analyzed in three runs. The three analytical runs were done on three different days. Samples were processed following the steps: (1) depletion; and (2) solution digestion, as described in Example 3, except that 10 μl of serum was depleted instead of 15 μl of serum. An internal standard was added to each sample. Samples were then tested following the same LC/MRM-MS condition as described in Example 3.

### Pilot Test

Twelve serum samples, including 8 normal serum samples and 6 colorectal cancer serum samples (2 stage II, 3 stage III and 1 stage IV), were run in triplicate. The disease-to-control ratios and the p-value for 13 proteins in the multiplex assay are listed in Table 9. The disease-to-control ratios and p-values of the selected peptides in the multiplex assay for 13 proteins at the peptide level are listed in Table 10.

TABLE 9
Multiplex pilot test result at protein level:
the disease-to-control ratio and p-value.

| | Ratio: | |
|---|---|---|
| Protein | Disease/control | p-value |
| ORM1 | 3.12 | 0.0008 |
| GSN | 0.34 | 0.0001 |
| C9 | 2.97 | 0.0001 |
| FN1 | 0.33 | 0.0003 |
| SERPINA3 | 2.84 | 0.000007 |
| PZP | 3.05 | 0.1086 |
| C2 | 1.34 | 0.0541 |
| PROZ | 0.72 | 0.1664 |
| PRG4 | 0.81 | 0.2014 |
| SAA2 | 52.60 | 0.0066 |
| CFHR2 | 0.68 | 0.0499 |
| LOC65387 | 0.97 | 0.8451 |
| HABP2 | 1.14 | 0.3621 |

TABLE 10
Multiplex pilot test result at peptide level:
the disease-to-control radio and p-value

| | | Ratio: | |
|---|---|---|---|
| | | Disease/ | |
| Protein | Peptide | Normal | p-Value |
| ORM1 | SEQ ID NO 1: | 2.50 | 0.0033156 |
| | WFYIASAFR | | |
| ORM1 | SEQ ID NO 2: | 2.47 | 0.0003729 |
| | TEDTIFLR | | |
| ORM1 | SEQ ID NO 3: | 3.87 | 0.0000128 |
| | SDVVYTDWK | | |
| ORM1 | SEQ ID NO 23: | 3.86 | 0.0010666 |
| | YVGGQEHFAHLLILR | | |
| GSN | SEQ ID NO 4: | 0.32 | 0.0000422 |
| | IFVWK | | |
| GSN | SEQ ID NO 5: | 0.32 | 0.0000399 |
| | QTQVSVLPEGGETPLFK | | |
| GSN | SEQ ID NO 6: | 0.34 | 0.0000089 |
| | AGALNSNDAFVLK | | |
| GSN | SEQ ID NO 24: | 0.32 | 0.0000097 |
| | HVVPNEVVVQR | | |
| GSN | SEQ ID NO 25: | 0.31 | 0.0000336 |
| | SEQCFILDHGK | | |
| C9 | SEQ ID NO 8: | 3.08 | 0.0000184 |
| | LSPIYNLVPVK | | |
| C9 | SEQ ID NO 9: | 3.24 | 0.0001148 |
| | AIEDYIEFSVR | | |
| C9 | SEQ ID NO 26: | 2.94 | 0.0000027 |
| | SIEVFGQFNGK | | |
| C9 | SEQ ID NO 27: | 2.79 | 0.00005551 |
| | TSNFNAAISLK | | |
| FN1 | SEQ ID NO 10: | 0.28 | 0.0000131 |
| | WLPSSSPVTGYR | | |
| FN1 | SEQ ID NO 11: | 0.28 | 0.0000457 |
| | IYLYTLNDNAR | | |
| FN1 | SEQ ID NO 12: | 0.30 | 0.0000578 |
| | SYTITGLQPGTDYK | | |
| FN1 | SEQ ID NO 28: | 0.33 | 0.0010085 |
| | VTWAPPPSILDTNFLVR | | |
| SERPINA3 | SEQ ID NO 13: | 3.24 | 0.0000512 |
| | EQLSLLDR | | |
| SERPINA3 | SEQ ID NO 15: | 2.94 | 0.0000022 |
| | ITLLSALVETR | | |
| SERPINA3 | SEQ ID NO 29: | 3.10 | 0.0000062 |
| | ADLSGITGAR | | |
| SERPINA3 | SEQ ID NO 30: | 2.61 | 0.0000085 |
| | AVLDVFEEGTEASAATAVK | | |
| PZP | SEQ ID NO 16: | 1.04 | 0.8121794 |
| | ATVLNYLPK | | |
| PZP | SEQ ID NO 17: | 7.97 | 0.1318286 |
| | AVGYLITGYQR | | |
| PZP | SEQ ID NO 31: | 7.44 | 0.1350599 |
| | SLFTDLVAEK | | |
| PZP | SEQ ID NO 32: | 1.07 | 0.7614844 |
| | NQGNTWLTAFVLK | | |
| PZP | SEQ ID NO 33: | 1.00 | 0.9896744 |
| | SSGSLLNNAIK | | |
| C2 | SEQ ID NO 18: | 1.40 | 0.0324419 |
| | HAFILQDTK | | |
| C2 | SEQ ID NO 19: | 1.39 | 0.0104918 |
| | AVISPGFDVFAK | | |
| C2 | SEQ ID NO 34: | 1.23 | 0.1953067 |
| | ECQGNGVWSGTEPICR | | |
| C2 | SEQ ID NO 35: | 1.29 | 0.1055677 |
| | EILNINQK | | |
| C2 | SEQ ID NO 36: | 1.32 | 0.0238423 |
| | DFHINLFR | | |
| PROZ | SEQ ID NO 20: | 0.79 | 0.3017053 |
| | GLLSGWAR | | |
| PROZ | SEQ ID NO 39: | 0.51 | 0.2008887 |
| | YSLWFK | | |
| PRG4 | SEQ ID NO 40: | 0.73 | 0.2066627 |
| | ITEVWGIPSPIDTVFTR | | |
| PRG4 | SEQ ID NO 41: | 0.75 | 0.1399844 |
| | GFGGLTGQIVAALSTAK | | |
| PRG4 | SEQ ID NO 42: | 0.93 | 0.7253475 |
| | IQYSPAR | | |
| PRG4 | SEQ ID NO 43: | 0.84 | 0.3454110 |
| | DQYYNIDVPSR | | |
| PRG4 | SEQ ID NO 44: | 0.67 | 0.0228997 |
| | CFESFER | | |
| SAA2 | SEQ ID NO 22: | 41.34 | 0.0017767 |
| | SFFSFLGEAFDGAR | | |
| SAA2 | SEQ ID NO 45: | 102.78 | 0.0095953 |
| | GPGGVWAAEAISDAR | | |
| SAA2 | SEQ ID NO 46: | 57.81 | 0.0012913 |
| | FFGHGAEDSLADQAANEWGR | | |
| CFHR2 | SEQ ID NO 47: | 1.76 | 0.0268359 |
| | ITCAEEGWSPTPK | | |
| CFHR2 | SEQ ID NO 48: | 0.28 | 0.0000001 |
| | GWSTPPK | | |
| CFHR2 | SEQ ID NO 49: | 1.30 | 0.3480817 |
| | TFDIVEFVCK | | |
| CFHR2 | SEQ ID NO 50: | 1.29 | 0.4772370 |
| | LVYPSCEK | | |
| LOC653879 | SEQ ID NO 51: | 0.82 | 0.4210371 |
| | IHWESASLLR | | |
| LOC653879 | SEQ ID NO 52: | 0.90 | 0.4952480 |
| | NTLIIYLDK | | |
| LOC653879 | SEQ ID NO 53: | 1.00 | 0.9992529 |
| | VYAYYNLEESCTR | | |
| LOC653879 | SEQ ID NO 54: | 0.96 | 0.7893764 |
| | ACEPGVDYVYK | | |
| LOC653879 | SEQ ID NO 55: | 0.87 | 0.4728866 |
| | TFISPIK | | |
| HABP2 | SEQ ID NO 56: | 1.10 | 0.9363291 |
| | FTCACPDQFK | | |
| HABP2 | SEQ ID NO 57: | 1.10 | 0.5591393 |
| | VVLGDQDLK | | |
| HABP2 | SEQ ID NO | 1.11 | 0.5277824 |
| | LIANTLCNSR | | |
| HABP2 | SEQ ID NO | 1.20 | 0.1554325 |
| | FLNWIK | | |

Among the 13-protein panel, the following 9 proteins have one or more peptides with a p-value less than 0.05: ORM1, GSN, C9, FN1, SERPINA3, C2, PRG4, SAA2, and CFHR2. Other than the peptides SEQ ID NO 23, SEQ ID NO 5, SEQ ID NO 25, SEQ ID NO 28, SEQ ID NO 31, SEQ ID NO 18, SEQ ID NO 34, SEQ ID NO 44, SEQ ID NO 48, and SEQ ID NO 53 in Table 8, 46 signature peptides were chosen from Table 8. The 92 peptides including 46 light peptides and 46 of heavy isotopes labeled peptides were ordered from Thermo-Fisher Scientific. The heavy isotopes are labeled on the C-terminus lysine or arginine.

1. A method for detecting colorectal cancer in a subject comprising:
obtaining a biological sample from a subject;
determining an expression level of the one or more biomarkers listed in Table 1 from the biological sample;
comparing the expression level of the one or more biomarkers with corresponding expression levels of the one or more biomarkers in a normal control sample; and
based on the comparison, determining the likelihood that the subject has colorectal cancer.

2. The method of claim 1, wherein the biological sample is selected from the group consisting of whole blood, blood plasma, serum, urine, tissue sample, cell sample, and tumor sample.

3. The method of claim 1, wherein the biological sample is serum.

4. The method of claim 1, wherein the expression level of one or more biomarkers in the biological sample from the subject shows a difference as compared with the expression level in the normal control sample, and wherein the difference detects the presence of colorectal cancer in the subject.

5. The method of claim 4, wherein the difference is increased and is at least 1.05 fold greater in the subject as compared with the normal control sample, and wherein the difference detects the presence of colorectal cancer the subject.

6. The method of claim 4, wherein the difference is decreased and is at least 0.9 less than the normal control sample, and wherein the difference detects the presence of colorectal cancer in the subject.

7. The method of claim 4, wherein one or more biomarkers is a protein.

8. The method of claim 7, wherein one or more protein biomarkers are selected from ORM1, GSN, C2 complement, C9 complement, PZP, CRP, CFHR1, CFHR2, SERPINA3, HABP2, CNDP1, CFHR2, SAA2, LOC653879 similar to Complement C3, PROZ, PON3, RARRES2, GGH, PRG4, MCAM1 and FN1.

9. The method of claim 7, wherein one or more protein biomarkers are selected from ORM1, GSN, C9, FN1, SERPINA3, PZP, C2, PROZ, PRG, and SAA2.

10. The method of claim 4, wherein the difference is determined by mass spectrometry, immunohistochemistry, ELISA, or Western blotting.

11. The method of claim 4, wherein the difference is determined by relative quantitative Multiple Reaction Monitoring (MRM-MS)-LC/MS/MS.

12. The method of claim 4, wherein the difference is determined by quantitative Multiple Reaction Monitoring (MRM-MS)-LC/MS/MS using stable isotope-labeled peptides corresponding to peptides derived from one or more biomarkers set forth in Table 1.

13. The method of claim 1, wherein one or more biomarkers is nucleic acid.

14. The method of claim 13, further comprising comparing the presence or absence, or the amount or concentration, of one or more nucleic acid biomarkers in the biological sample with the presence or absence, or the amount or concentration, of one or more nucleic acid biomarkers in the normal control sample.

15. The method of claim 14, wherein the detecting of the presence or absence, or the amount or concentration of, one or more nucleic acid biomarkers is carried out by RT-PCR.

16. (canceled)

17. A kit for detecting colorectal cancer by comparing the presence or absence, or the amount or concentration, of one or more nucleic acid biomarkers in the biological sample with the presence or absence, or the amount or concentration, of one or more nucleic acid biomarkers in the normal control sample, comprising an mRNA extracting buffer, at least one reverse transcription enzyme, at least one pair of primers that have the nucleotide sequences encompassed coding for any one or more of the amino acid residues of the protein biomarkers listed in Table 1, and instructions for use.

18. A kit for detecting colorectal cancer by comparing the amount or concentration, of one or more biomarkers listed in table 1 in the biological sample with the amount or concentration, of one or more biomarkers in the normal control sample, comprising isotopically labeled peptides corresponding to peptides derived from one or more biomarkers in Table 1, an internal standard, a set of calibrators, and instruction for use.

19. The method of claim 1, wherein one or more biomarkers further comprises CEA.

20. The method of claim 1, wherein one or more biomarkers further comprises CEA and carbohydrate antigen 19-9.


SEQUENCE LISTING (116 sequences)

SEQ ID NO: 1 - protein, 9 aa, human
WFYIASAFR

SEQ ID NO: 2 - protein, 8 aa, human
TEDTIFLR

SEQ ID NO: 3 - protein, 8 aa, human
SDWYTDWK

SEQ ID NO: 4 - protein, 5 aa, human
IFVWK

SEQ ID NO: 5 - protein, 17 aa, human
QTQVSVLPEGGETPLFK

SEQ ID NO: 6 - protein, 13 aa, human
AGALNSNDAFVLK

SEQ ID NO: 7 - protein, 6 aa, human
YAFELK

SEQ ID NO: 8 - protein, 11 aa, human
LSPIYNLVPVK

SEQ ID NO: 9 - protein, 11 aa, human
AIEDYIEFSVR

SEQ ID NO: 10 - protein, 12 aa, human
WLPSSSPVTGYR

SEQ ID NO: 11 - protein, 11 aa, human
IYLYTLNDNAR

SEQ ID NO: 12 - protein, 14 aa, human
SYTITGLQPGTDYK

SEQ ID NO: 13 - protein, 8 aa, human
EQLSLLDR

SEQ ID NO: 14 - protein, 9 aa, human
EIGELYLPK

SEQ ID NO: 15 - protein, 11 aa, human
ITLLSALVETR

SEQ ID NO: 16 - protein, 9 aa, human
ATVLNYLPK

SEQ ID NO: 17 - protein, 11 aa, human
AVGYLITGYQR

SEQ ID NO: 18 - protein, 9 aa, human
HAFILQDTK

SEQ ID NO: 19 - protein, 12 aa, human
AVISPGFDVFAK

SEQ ID NO: 20 - protein, 8 aa, human
GLLSGWAR

SEQ ID NO: 21 - protein, 11 aa, human
AIGPSQTHTIR

SEQ ID NO: 22 - protein, 14 aa, human
SFFSFLGEAFDGAR

SEQ ID NO: 23 - protein, 15 aa, human
YVGGQEHFAHLLILR

SEQ ID NO: 24 - protein, 11 aa, human
HVVPNEVVVQR

SEQ ID NO: 25 - protein, 11 aa, human
SEDCFILDHGK

SEQ ID NO: 26 - protein, 11 aa, human
SIEVFGQFNGK

SEQ ID NO: 27 - protein, 11 aa, human
TSNFNAAISLK

SEQ ID NO: 28 - protein, 17 aa, human
VTWAPPPSIDLTNFLVR

SEQ ID NO: 29 - protein, 10 aa, human
ADLSGITGAR

SEQ ID NO: 30 - protein, 19 aa, human
AVLDVFEEGTEASAATAVK

SEQ ID NO: 31 - protein, 10 aa, human
SLFTDLVAEK

SEQ ID NO: 32 - protein, 13 aa, human
NQGNTWLTAFVLK

SEQ ID NO: 33 - protein, 11 aa, human
SSGSLLNNAIK

SEQ ID NO: 34 - protein, 16 aa, human
ECQGNGVWSGTEPICR

SEQ ID NO: 35 - protein, 8 aa, human
EILNINQK

SEQ ID NO: 36 - protein, 8 aa, human
DFHINLFR

SEQ ID NO: 37 - protein, 12 aa, human
APDLQDLPWQVK

SEQ ID NO: 38 - protein, 9 aa, human
ENFVLTTAK

SEQ ID NO: 39 - protein, 6 aa, human
YSLWFK

SEQ ID NO: 40 - protein, 17 aa, human
ITEVWGIPSPIDTVFTR

SEQ ID NO: 41 - protein, 17 aa, human
GFGGLTGQIVAALSTAK

SEQ ID NO: 42 - protein, 7 aa, human
IQYSPAR

SEQ ID NO: 43 - protein, 11 aa, human
DQYYNIDVPSR

SEQ ID NO: 44 - protein, 7 aa, human
CFESFER

SEQ ID NO: 45 - protein, 15 aa, human
GPGGVWAAEAISDAR

SEQ ID NO: 46 - protein, 20 aa, human
FFGHGAEDSLADQAANEWGR

SEQ ID NO: 47 - protein, 13 aa, human
ITCAEEGWSPTPK

SEQ ID NO: 48 - protein, 7 aa, human
GWSTPPK

SEQ ID NO: 49 - protein, 10 aa, human
TGDIVEFVCK

SEQ ID NO: 50 - protein, 9 aa, human
LVYPSCEEK

SEQ ID NO: 51 - protein, 10 aa, human
IHWESASLLR

SEQ ID NO: 52 - protein, 9 aa, human
NTLIIYLDK

SEQ ID NO: 53 - protein, 13 aa, human
VYAYYNLEESCTR

SEQ ID NO: 54 - protein, 11 aa, human
ACEPGVDYVYK

SEQ ID NO: 55 - protein, 7 aa, human
TFISPIK

SEQ ID NO: 56 - protein, 10 aa, human
FTCACPDQFK

SEQ ID NO: 57 - protein, 9 aa, human
VVLGDQDLK

SEQ ID NO: 58 - protein, 10 aa, human
LIANTLCNSR

SEQ ID NO: 59 - protein, 6 aa, human
FLNWIK

SEQ ID NO: 60 - protein, 201 aa, HUMAN
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 61 - protein, 782 aa, human
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 62 - protein, 752 aa, HUMAN
MGPLMVLFCLLFLYPGLADSAPSCPQNVNISGGTFTLSHGWAPGSLLTYSCPQGLYPSPASRLCKSSGQWQTPGATRSLSKAVCKPVRCPAPVSFENGIYTPRLGSYPVGGNVSFECEDGFILRGSPVRQCRPNGMWDGETAVCDNGAGHCPNPGISLGAVRTGFRFGHGDKVRYRCSSNLVLTGSSERECQGNGVWSGTEPICRQPYSYDFPEDVAPALGTSFSHMLGATNPTQKTKESLGRKIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 63 - protein, 559 aa, HUMAN
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 64 - protein, 1424 aa, HUMAN
MRKDRLLHLCLVLLLILLSASDSNSTEPQYMVLVPSLLHTEAPKKGCVLLSHLNETVTVSASLESGRENRSLFTDLVAEKDLFHCVSFTLPRISASSEVAFLSIQIKGPTQDFRKRNTVLVLNTQSLVFVQTDKPMYKPGQTVRFRVVSVDENFRPRNELIPLIYLENPRRNRIAQWQSLKLEAGINQLSFPLSSEPIQGSYRVVVQTESGGRIQHPFTVEEFVLPKFEVKVQVPKIISIMDEKVNITVCGEYTYGKPVPGLATVSLCRKLSRVLNCDKQEVCEEFSQQLNSNGCITQQVHTKMLQITNTGFEMKLRVEARIREEGTDLEVTANRISEITNIVSKLKFVKVDSHFRQGIPFFAQVLLVDGKGVPIPNKLFFISVNDANYYSNATTNEQGLAQFSINTTSISVNKLFVRVFTVHPNLCFHYSWVAEDHQGAQHTANRVFSLSGSYIHLEPVAGTLPCGHTETITAHYTLNRQAMGELSELSFHYLIMAKGVIVRSGTHTLPVESGDMKGSFALSFPVESDVAPIARMFIFAILPDGEVVGDSEKFEIENCLANKVDLSFSPAQSPPASHAHLQVAAAPQSLCALRAVDQSVLLMKPEAELSVSSVYNLLTVKDLTNFPDNVDQQEEEQGHCPRPFFIHNGAIYVPLSSNEADIYSFLKGMGLKVFTNSKIRKPKSCSVIPSVSAGAVGQGYYGAGLGVVERPYVPQLGTYNVIPLNNEQSSGPVPETVRSYFPETWIWELVAVNSSGVAEVGVTVPDTITEWKAGAFCLSEDAGLGISSTASLRAFQPFFVELTMPYSVIRGEVFTLKATVLNYLPKCIRVSVQLKASPAFLASQNTKGEESYCICGNERQTLSWTVTPKTLGNVNFSVSAEAMQSLELCGNEVVEVPEIKRKDTVIKTLLVEAEGIEQEKTFSSMTCASGANVSEQLSLKLPSNVVKESARASFSVLGDILGSAMQNIQNLLQMPYGCGEQNMVLFAPNIYVLNYLNETQQLTQEIKAKAVGYLITGYQRQLNYKHQDGSYSTFGERYGRNQGNTWLTAFVLKTFAQARSYIFIDEAHITQSLTWLSQMQKDNGCFRSSGSLLNNAIKGGVEDEATLSAYVTIALLEIPLPVTNPIVRNALFCLESAWNVAKEGTHTQAPSAEVEMTSYVLLAYLTAQPAPTSGDLTSATNIVKWIMKQQNAQGGFSSTQDTVVALHALSRYGAATFTRTEKTAQVTVQDSQTFSTNFQVDNNNLLLLQQISLPELPGEYVITVTGERCVYLQTSMKYNILPEKEDSPFALKVQTVPQTCDGHKAHTSFQISLTISYTGNRPASNMVIVDVKMVSGFIPLKPTVKMLERSSSVSRTEVSNNHVLIYVEQVTNQTLSFSFMVLQDIPVGDLKPAIVKVYDYYETDESVVAEYIAPCSTDTEHGNV

SEQ ID NO: 65 - protein, 224 aa, HUMAN
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 66 - protein, 330 aa, HUMAN
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 67 - protein, 423 aa, HUMAN
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 68 - protein, 560 aa, HUMAN
MFARMSDLHVLLLMALVGKTACGFSLMSLLESLDPDWTPDQYDYSYEDYNQEENTSSTLTHAENPDWYYTEDQADPCQPNPCEHGGDCLVHGSTFTCSCLAPFSGNKCQKVQNTCKDNPCGRGQCLITQSPPYYRCVCKHPYTGPSCSQVVPVCRPNPCQNGATCSRHKRRSKFTCACPDQFKGKFCEIGSDDCYVGDGYSYRGKMNRTVNQHACLYWNSHLLLQENYNMFMEDAETHGIGEHNFCRNPDADEKPWCFIKVTNDKVKWEYCDVSACSAQDVAYPEESPTEPSTKLPGFDSCGKTEIAERKIKRIYGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDIALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIKSESGF

SEQ ID NO: 69 - protein, 507 aa, HUMAN
MDPKLGRMAASLLAVLLLLLERGMFSSPSPPPALLEKVFQYIDLHQDEFVQTLKEWVAIESDSVQPVPRFRQELFRMMAVAADTLQRLGARVASVDMGPQQLPDGQSLPIPPVILAELGSDPTKGTVCFYGHLDVQPADRGDGWLTDPYVLTEVDGKLYGRGATDNKGPVLAWINAVSAFRALEQDLPVNIKFIIEGMEEAGSVALEELVEKEKDRFFSGVDYIVISDNLWISQRKPAITYGTRGNSYFMVEVKCRDQDFHSGTFGGILHEPMADLVALLGSLVDSSGHILVPGIYDEVVPLTEEEINTYKAIHLDLEEYRNSSRVEKFLFDTKEEILMHLWRYPSLSIHGIEGAFDEPGTKTVIPGRVIGKFSIRLVPHMNVSAVEKQVTRHLEDVFSKRNSSNKMVVSMTLGLHPWIANIDDTQYLAAKRAIRTVFGTEPDMIRDGSTIPIAKMFQEIVHKSVVLIPLGAVDDGEHSQNEKINRWNYIEGTKLFAAFFLEMAQLH

SEQ ID NO: 70 - protein, 270 aa, HUMAN
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 71 - protein, 122 aa, HUMAN
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 72 - protein, 400 aa, HUMAN
MAGCVPLLQGLVLVLALHRVEPSVFLPASKANDVLVRWKRAGSYLLEELFEGNLEKECYEEICVYEEAREVFENEVVTDEFWRRYKGGSPCISQPCLHNGSCQDSIWGYTCTCSPGYEGSNCELAKNECHPERTDGCQHFCLPGQESYTCSCAQGYRLGEDHKQCVPHDQCACGVLTSEKRAPDLQDLPWQVKLTNSEGKDFCGGVIIRENFVLTTAKCSLLHRNITVKTYFNRTSQDPLMIKITHVHVHMRYDADAGENDLSLLELEWPIQCPGAGLPVCTPEKDFAEHLLIPRTRGLLSGWARNGTDLGNSLTTRPVTLVEGEECGQVLNVTVTTRTYCERSSVAAMHWMDGSVVTREHRGSWFLTGVLGSQPVGGQAHMVLVTKVSRYSLWFKQIMN

SEQ ID NO: 73 - protein, 354 aa, HUMAN
MGKLVALVLLGVGLSLVGEMFLAFRERVNASREVEPVEPENCHLIEELESGSEDIDILPSGLAFISSGLKYPGMPNFAPDEPGKIFLMDLNEQNPRAQALEISGGFDKELFNPHGISIFIDKDNTVYLYVVNHPHMKSTVEIFKFEEQQRSLVYLKTIKHELLKSVNDIVVLGPEQFYATRDHYFTNSLLSFFEMILDLRWTYVLFYSPREVKVVAKGFCSANGITVSADQKYVYVADVAAKNIHIMEKHDNWDLTQLKVIQLGTLVDNLTVDPATGDILAGCHPNPMKLLNYNPEDPPGSEVLRIQNVLSEKPRVSTVYANNGSVLQGTSVASVYHGKILIGTVFHKTLYCEL

SEQ ID NO: 74 - protein, 163 aa, HUMAN
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 75 - protein, 318 aa, HUMAN
MASPGCLLCVLGLLLCGAASLELSRPHGDTAKKPIIGILMQKCRNKVMKNYGRYYIAASYVKYLESAGARVVPVRLDLTEKDYEILFKSINGILFPGGSVDLRRSDYAKVAKIFYNLSIQSFDDGDYFPVWGTCLGFEELSLLISGECLLTATDTVDVAMPLNFTGGQLHSRMFQNFPTELLLSLAVEPLTANFHKWSLSVKNFTMNEKLKKFFNVLTTNTDGKIEFISTMEGYKYPVYGVQWHPEKAPYEWKNLDGISHAPNAVKTAFYLAEFFVNEARKNNHHFKSESEEEKALIYQFSPIYTGNISSFQQCYIFD

SEQ ID NO: 76 - protein, 1404 aa, HUMAN
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 77 - protein, 646 aa, HUMAN
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 78 - protein, 2296 aa, HUMAN
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTTIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 79 - protein, 388 aa, HUMAN
MVFQALAQYQKDAPDHQELNLDVSLQLPSRSSKITHRIHWESASLLRSEETKENEGFTVTEGKGQGTLSVVTMYHAKAKDQLTCNKFDLKVTIKPAPETEKRPQDAKNTMILEICTRYRDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 80 - DNA, 1404 bp, HUMAN
cttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtctttgggtgccccaactgaccacacccccattccatgaacctacagaga

SEQ ID NO: 81 - DNA, 847 bp, HUMAN
acagagtaaacttttgctgggctccaagtgaccgcccatagtttattataaaggtgactgcaccctgcagccaccagcactgcctggctccacgtgcctcctggtctcagtatggcgctgtcctgggttcttacagtcctgagcctcctacctctgctggaagcccagatcccattgtgtgccaacctagtaccggtgcccatcaccaacgccaccctggaccggatcactggcaagtggttttatatcgcatcggcctttcgaaacgaggagtacaataagtcggttcaggagatccaagcaaccttcttttacttcacccccaacaagacagaggacacgatctttctcagagagtaccagacccgacaggaccagtgcatctataacaccacctacctgaatgtccagcgggaaaatgggaccatctccagatacgtgggaggccaagagcatttcgctcacttgctgatcctcagggacaccaagacctacatgcttgcttttgacgtgaacgatgagaagaactgggggctgtctgtctatgctgacaagccagagacgaccaaggagcaactgggagagttctacgaagctctcgactgcttgcgcattcccaagtcagatgtcgtgtacaccgattggaaaaaggataagtgtgagccactggagaagcagcacgagaaggagaggaaacaggaggagggggaatcctagcaggacacagccttggatcaggacagagacttgggggccatcctgcccctccaacccgacatgtgtacctcagctttttccctcacttgcatcaataaagcttctgtgtttggaacagctaaaaaaaaaa

SEQ ID NO: 82 - DNA, 803 bp, HUMAN
gcccatagtttattataaaggtgactgcaccctgcagccaccagcactgcctggctccacgtgcctcctggtctcagtatggcgctgtcctgggttcttacagtcctgagcctcctacctctgctggaagcccagatcccattgtgtgccaacctagtaccggtgcccatcaccaacgccaccctggaccagatcactggcaagtggttttatatcgcatcggcctttcgaaacgaggagtacaataagtcggttcaggagatccaagcaaccttcttttacttcacccccaacaagacagaggacacgatctttctcagagagtaccagacccgacaggaccagtgcatctataacaccacctacctgaatgtccagcgggaaaatgggaccatctccagatacgtgggaggccaagagcatttcgctcacttgctgatcctcagggacaccaagacctacatgcttgcttttgacgtgaacgatgagaagaactgggggctgtctgtctatgctgacaagccagagacgaccaaggagcaactgggagagttctacgaagctctcgactgcttgcgcattcccaagtcagatgtcgtgtacaccgattggaaaaaggataagtgtgagccactggagaagcagcacgagaaggagaggaaacaggaggagggggaatcctagcaggacacagccttggatcaggacagagacttggggccatcctgcccctccaacccgacatgtgtacctcagctttttccctcacttgcatcaataaagcttctgtgtttggaacagct

SEQ ID NO: 83 - DNA, 1404 bp, HUMAN; identical to SEQ ID NO: 80

SEQ ID NO: 84 - DNA, 2719 bp, HUMAN
acttaaggtcggcgacccgaggccgcggctgccgactgggtcccctgccgctgtcgccaccatggctccgcaccgccccgcgcccgcgctgctttgcgcgctgtccctggcgctgtgcgcgctgtcgctgcccgtccgcgcggccactgcgtcgcggggggcgtcccaggcgggggcgccccaggggcgggtgcccgaggcgcggcccaacagcatggtggtggaacaccccgagttcctcaaggcagggaaggagcctggcctgcagatctggcgtgtggagaagttcgatctggtgcccgtgcccaccaacctttatggagacttcttcacgggcgacgcctacgtcatcctgaagacagtgcagctgaggaacggaaatctgcagtatgacctccactactggctgggcaatgagtgcagccaggatgagagcggggcggccgccatctttaccgtgcagctggatgactacctgaacggccgggccgtgcagcaccgtgaggtccagggcttcgagtcggccaccttcctaggctacttcaagtctggcctgaagtacaagaaaggaggtgtggcatcaggattcaagcacgtggtacccaacgaggtggtggtgcagagactcttccaggtcaaagggcggcgtgtggtccgtgccaccgaggtacctgtgtcctgggagagcttcaacaatggcgactgcttcatcctggacctgggcaacaacatccaccagtggtgtggttccaacagcaatcggtatgaaagactgaaggccacacaggtgtccaagggcatccgggacaacgagcggagtggccgggcccgagtgcacgtgtctgaggagggcactgagcccgaggcgatgctccaggtgctgggccccaagccggctctgcctgcaggtaccgaggacaccgccaaggaggatgcggccaaccgcaagctggccaagctctacaaggtctccaatggtgcagggaccatgtccgtctccctcgtggctgatgagaaccccttcgcccagggggccctgaagtcagaggactgcttcatcctggaccacggcaaagatgggaaaatctttgtctggaaaggcaagcaggcaaacacggaggagaggaaggctgccctcaaaacagcctctgacttcatcaccaagatggactaccccaagcagactcaggtctcggtccttcctgagggcggtgagaccccactgttcaagcagttcttcaagaactggcgggacccagaccagacagatggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctgaggaggggcagggcccacccatgtcaccggtcagtgccttttggaactgtccttccctcaaagaggccttagagcgagcagagcagctctgctatgagtgtgtgtgtgtgtgtgtgttgtttcttttttttttttttacagtatccaaaaatagccctgcaaaaattcagagtccttgcaaaattgtctaaaatgtcagtgtttgggaaattaaatccaataaaaacattttgaagtgtgaaaaaaaaaaaaaaaaaaaaaaaaaaaaaaaaaaaaaaaaaaaaaaaaaaaaaaaaaaaaaaaaaaaaaa

SEQ ID NO: 85 - DNA, 2602 bp, HUMAN
gccgtgtcgccaccatggctccgcaccgccccgcgcccgcgctgctttgcgcgctgtccctggcgctgtgcgcgctgtcgctgcccgtccgcgcggccactgcgtcgcggggggcgtcccaggcgggggcgccccaggggcgggtgcccgaggcgcggcccaacagcatggtggtggaacaccccgagttcctcaaggcagggaaggagcctggcctgcagatctggcgtgtggagaagttcgatctggtgcccgtgcccaccaacctttatggagacttcttcacgggcgacgcctacgtcatcctgaagacagtgcagctgaggaacggaaatctgcagtatgacctccactactggctgggcaatgagtgcagccaggatgagagcggggcggccgccatctttaccgtgcagctggatgactacctgaacggccgggccgtgcagcaccgtgaggtccagggcttcgagtcggccaccttcctaggctacttcaagtctggcctgaagtacaagaaaggaggtgtggcatcaggattcaagcacgtggtacccaacgaggtggtggtgcagagactcttccaggtcaaagggcggcgtgtggtccgtgccaccgaggtacctgtgtcctgggagagcttcaacaatggcgactgcttcatcctggacctgggcaacaacatccaccagtggtgtggttccaacagcaatcggtatgaaagactgaaggccacacaggtgtccaagggcatccgggacaacgagcggagtggccgggcccgagtgcacgtgtctgaggagggcactgagcccgaggcgatgctccaggtgctgggccccaagccggctctgcctgcaggtaccgaggacaccgccaaggaggatgcggccaaccgcaagctggccaagctctacaaggtctccaatggtgcagggaccatgtccgtctccctcgtggctgatgagaaccccttcgcccagggggccctgaagtcagaggactgcttcatcctggaccacggcaaagatgggaaaatctttgtctggaaaggcaagcaggcaaacacggaggagaggaaggctgccctcaaaacagcctctgacttcatcaccaagatggactaccccaagcagactcaggtctcggtccttcctgagggcggtgagaccccactgttcaagcagttcttcaagaactggcgggacccagaccagacagatggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctgaggaggggcagggcccacccatgtcaccggtcagtgccttttggaactgtccttccctcaaagaggccttagagcgagcagagcagctctgctatgagtgtgtgtgtgtgtgtgtgttgtttcttttttttttttttacagtatccaaaaatagccctgcaaaaattcagagtccttgcaaaattgtctaaaatgtcagtgtttgggaaattaaatccaataaaaacattttgaagtgtg

SEQ ID NO: 86 - DNA, 2609 bp, HUMAN
ggctctctacctctcgccgcccctagggaggacaccatgggcccactgatggttcttttttgcctgctgttcctgtacccaggtctggcagactcggctccctcctgccctcagaacgtgaatatctcgggtggcaccttcaccctcagccatggctgggctcctgggagccttctcacctactcctgcccccagggcctgtacccatccccagcatcacggctgtgcaagagcagcggacagtggcagaccccaggagccacccggtctctgtctaaggcggtctgcaaacctgtgcgctgtccagcccctgtctcctttgagaatggcatttataccccacggctggggtcctatcccgtgggtggcaatgtgagcttcgagtgtgaggatggcttcatattgcggggctcgcctgtgcgtcagtgtcgccccaacggcatgtgggatggagaaacagctgtgtgtgataatggggctggccactgccccaacccaggcatttcactgggcgcagtgcggacaggcttccgctttggtcatggggacaaggtccgctatcgctgctcctcgaatcttgtgctcacggggtcttcggagcgggagtgccagggcaacggggtctggagtggaacggagcccatctgccgccaaccctactcttatgacttccctgaggacgtggcccctgccctgggcacttccttctcccacatgcttggggccaccaatcccacccagaagacaaaggaaagcctgggccgtaaaatccaaatccagcgctctggtcatctgaacctctacctgctcctggactgttcgcagagtgtgtcggaaaatgactttctcatcttcaaggagagcgcctccctcatggtggacaggatcttcagctttgagatcaatgtgagcgttgccattatcacctttgcctcagagcccaaagtcctcatgtctgtcctgaacgacaactcccgggatatgactgaggtgatcagcagcctggaaaatgccaactataaagatcatgaaaatggaactgggactaacacctatgcagccttaaacagtgtctatctcatgatgaacaaccaaatgcgactcctcggcatggaaacgatggcctggcaggaaatccgacatgccatcatccttctgacagatggaaagtccaatatgggtggctctcccaagacagctgttgaccatatcagagagatcctgaacatcaaccagaagaggaatgactatctggacatctatgccatcggggtgggcaagctggatgtggactggagagaactgaatgagctagggtccaagaaggatggtgagaggcatgccttcattctgcaggacacaaaggctctgcaccaggtctttgaacatatgctggatgtctccaagctcacagacaccatctgcggggtggggaacatgtcagcaaacgcctctgaccaggagaggacaccctggcatgtcactattaagcccaagagccaagagacctgccggggggccctcatctccgaccaatgggtcctgacagcagctcattgcttccgcgatggcaacgaccactccctgtggagggtcaatgtgggagaccccaaatcccagtggggcaaagaattccttattgagaaggcggtgatctccccagggtttgatgtctttgccaaaaagaaccagggaatcctggagttctatggtgatgacatagctctgctgaagctggcccagaaagtaaagatgtccacccatgccaggcccatctgccttccctgcacgatggaggccaatctggctctgcggagacctcaaggcagcacctgtagggaccatgagaatgaactgctgaacaaacagagtgttcctgctcattttgtcgccttgaatgggagcaaactgaacattaaccttaagatgggagtggagtggacaagctgtgccgaggttgtctcccaagaaaaaaccatgttccccaacttgacagatgtcagggaggtggtgacagaccagttcctatgcagtgggacccaggaggatgagagtccctgcaagggagaatctgggggagcagttttccttgagcggagattcaggttttttcaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctctagccatggccactgagccctctgctgccctgccagaatctgccgcccctccatcttctacctctgaatggccacccttagaccctgtgatccatcctctctcctagctgagtaaatccgggtctctaggatgccagaggcagcgcacacaagctgggaaatcctcagggctcctaccagcaggactgcctcgctgccccacctcccgctccttggcctgtccccagattccttccctggttgacttgactcatgcttgtttcactttcacatggaatttcccagttatgaaattaataaaaatcaatggtttccac

SEQ ID NO: 87 - DNA, 2693 bp, HUMAN
gcttgttccctgtcctctggccctttgcaaataaatgccttaccagacctgccctgccaccccactcgcagccacccagcaagagcagcatgtcagcctgccggagctttgcagttgcaatctgcattttagaaataagcatcctcacagcacagtacacgaccagttatgacccagagctaacagaaagcagtggctctgcatcacacatagactgcagaatgagcccctggagtgaatggtcacaatgcgatccttgtctcagacaaatgtttcgttcaagaagcattgaggtctttggacaatttaatgggaaaagatgcaccgacgctgtgggagacagacgacagtgtgtgcccacagagccctgtgaggatgctgaggatgactgcggaaatgactttcaatgcagtacaggcagatgcataaagatgcgacttcggtgtaatggtgacaatgactgcggagacttttcagatgaggatgattgtgaaagtgagccccgtcccccctgcagagacagagtggtagaagagtctgagctggcacgaacagcaggctatgggatcaacattttagggatggatcccctaagcacaccttttgacaatgagttctacaatggactctgtaaccgggatcgggatggaaacactctgacatactaccgaagaccttggaacgtggcttctttgatctatgaaaccaaaggcgagaaaaatttcagaaccgaacattacgaagaacaaattgaagcatttaaaagtatcatccaagagaagacatcaaattttaatgcagctatatctctaaaatttacacccactgaaacaaataaagctgaacaatgttgtgaggaaacagcctcctcaatttctttacatggcaagggtagttttcggttttcatattccaaaaatgaaacttaccaactatttttgtcatattcttcaaagaaggaaaaaatgtttctgcatgtgaaaggagaaattcatctgggaagatttgtaatgagaaatcgcgatgttgtgctcacaacaacttttgtggatgatataaaagctttgccaactacctatgaaaagggagaatattttgcctttttggaaacctatggaactcactacagtagctctgggtctctaggaggactctatgaactaatatatgttttggataaagcttccatgaagcggaaaggtgttgaactaaaagacataaagagatgccttgggtatcatctggatgtatctctggctttctctgaaatctctgttggagctgaatttaataaagatgattgtgtaaagaggggagagggtagagctgtaaacatcaccagtgaaaacctcatagatgatgttgtttcactcataagaggtggaaccagaaaatatgcatttgaactgaaagaaaagcttctccgaggaaccgtgattgatgtgactgactttgtcaactgggcctcttccataaatgatgctcctgttctcattagtcaaaaactgtctcctatatataatctggttccagtgaaaatgaaaaatgcacacctaaagaaacaaaacttggaaagagccattgaagactatatcaatgaatttagtgtaagaaaatgccacacatgccaaaatggaggtacagtgattctaatggatggaaagtgtttgtgtgcctgcccattcaaatttgagggaattgcctgtgaaatcagtaaacaaaaaatttctgaaggattgccagccctagagttccccaatgaaaaatagagctgttggcttctctgagctccagtggaagaagaaaacactagtaccttcagatcctacccctgaagataatcttagctgccaagtaaatagcaacatgcttcatgaaaatcctaccaacctctgaagtctcttctctcttaggtctataatttttttttaaatttttcttccttaaactcctgtgatgtttccattttttgttccctaatgagaagtcaacagtgaaatacgccagaactgctttatcccacggaaaatgccaatctcttctaaaaaaaaacaaaattaaattaaaaacagaatgttggtttaaaaaacttcaaagtaattttcaaacggctttgtatggttaacatattctgccaggtccatgaccacacgtctgtaccatgcaatttaactcttatttacattgttatgtttagtttggttatttgcttaggtgtgcatacattcattcagcaaatgctgagcaccagccacgtgcacagcagttgcttttactagtcttagctctacgatttaaatccatgtgtccaagggggaaaacatattatatttgtaaccaaaaactactagtttaccagaggactgaagggagataaagaggagttggttaatgggtacaaaaatccagttagatgaaaggaataatatagatagtgttcagtagcagaatagaatgaacataaactattagtttaaattatgtgaaattccttctatttgatcatattttacaagaaaaaacatcaattttatatagtccaacttaatacctagccttatgagttgtataaggtaaggttacctacctgagaagctgattaacattggttgtacaatcttattcattagagaacatggtgcttagggtctgagaccttttgaaaggtctgagaactctttaaaaaaaggaaa

SEQ ID NO: 88 - DNA, 2026 bp, HUMAN
cagcatgtcagcctgccggagctttgcagttgcaatctgcattttagaaataagcatcctcacagcacagtacacgaccagttatgacccagagctaacagaaagcagtggctctgcatcacacatagactgcagaatgagcccctggagtgaatggtcacaatgcgatccttgtctcagacaaatgtttcgttcaagaagcattgaggtctttggacaatttaatgggaaaagatgcaccgacgctgtgggagacagacgacagtgtgtgcccacagagccctgtgaggatgctgaggatgactgcggaaatgactttcaatgcagtacaggcagatgcataaagatgcgacttcggtgtaatggtgacaatgactgcggagacttttcagatgaggatgattgtgaaagtgagccccgtcccccctgcagagacagagtggtagaagagtctgagctggcacgaacagcaggctatgggatcaacattttagggatggatcccctaagcacaccttttgacaatgagttctacaatggactctgtaaccgggatcgggatggaaacactctgacatactaccgaagaccttggaacgtggcttctttgatctatgaaaccaaaggcgagaaaaatttcagaaccgaacattacgaagaacaaattgaagcatttaaaagtatcatccaagagaagacatcaaattttaatgcagctatatctctaaaatttacacccactgaaacaaataaagctgaacaatgttgtgaggaaacagcctcctcaatttctttacatggcaagggtagttttcggttttcatattccaaaaatgaaacttaccaactatttttgtcatattcttcaaagaaggaaaaaatgtttctgcatgtgaaaggagaaattcatctgggaagatttgtaatgagaaatcgcgatgttgtgctcacaacaacttttgtggatgatataaaagctttgccaactacctatgaaaagggagaatattttgcctttttggaaacctatggaactcactacagtagctctgggtctctaggaggactctatgaactaatatatgttttggataaagcttccatgaagcggaaaggtgttgaactaaaagacataaagagatgccttgggtatcatctggatgtatctctggctttctctgaaatctctgttggagctgaatttaataaagatgattgtgtaaagaggggagagggtagagctgtaaacatcaccagtgaaaacctcatagatgatgttgtttcactcataagaggtggaaccagaaaatatgcatttgaactgaaagaaaagcttctccgaggaaccgtgattgatgtgactgactttgtcaactgggcctcttccataaatgatgctcctgttctcattagtcaaaaactgtctcctatatataatctggttccagtgaaaatgaaaaatgcacacctaaagaaacaaaacttggaaagagccattgaagactatatcaatgaatttagtgtaagaaaatgccacacatgccaaaatggaggtacagtgattctaatggatggaaagtgtttgtgtgcctgcccattcaaatttgagggaattgcctgtgaaatcagtaaacaaaaaatttctgaaggattgccagccctagagttccccaatgaaaaatagagctgttggcttctctgagctccagtggaagaagaaaacactagtaccttcagactcctacccctgaagataatcttagctgccaagtaaatagcaacatgcttcatgaaaatcctaccaacctctgaagtctcttctctcttaggtctataattttttttttaatttttcttccttaaactcctgtgatgtttccattttttgttccctaatgagaagtcaacagtgaaatacgccagaactgctttatcccacggaaaatgccaatctcttctaaaaaaaaacaaaattaaattaaaaacagaatgttggtttaaaaaacttcaaagaaaaaaaaaaaaa

SEQ ID NO: 89 - DNA, 4615 bp, HUMAN
ggacacaaccctgagatttatccctcacaatgcggaaagacagacttcttcatttatgtcttgtgctacttcttatcctgctttctgccagtgactcaaactctacagaaccgcagtatatggtgctggtcccctccctgctccacactgaggcccctaagaagggctgtgtccttctgagccacctgaatgagacagtgactgtaagtgcttccttggagtctggcagggaaaacaggagcctcttcactgacctggtggcggagaaggacttattccactgtgtctccttcactctcccaaggatctcagcctcttcagaggtggcattccttagcatccagataaaggggcctacgcaagatttcaggaagaggaacacagttctggtactgaacacccaaagtctggtctttgtccagacagacaaacccatgtataaaccaggacagacagtaagattccgtgttgtctccgtggatgaaaattttcgccctcgaaatgaactgattccactgatataccttgagaacccaagaagaaatcgaattgcacaatggcagagtctcaagctagaagctggcatcaatcagttgtcctttcccctctcatcagagcccattcagggctcctacagggtggtggtacagacagaatcaggtggaaggatacagcaccccttcaccgtggaggaatttgtgcttcccaagtttgaggtcaaagttcaggtgccaaagataatcagtatcatggatgaaaaagtgaacataacagtctgtggagaatacacttatgggaagcctgtcccaggacttgcaactgtgagcctgtgtagaaaattatctcgtgttcttaattgtgacaagcaggaggtctgtgaggaattcagtcaacagcttaacagcaatggctgcatcacccaacaagtacacaccaaaatgctccagattacaaatacgggctttgaaatgaagcttagagtggaagccaggatcagagaagaggggacagacctggaagtcactgcaaacaggatcagtgaaatcacaaacattgtatccaaactcaaattcgtgaaagtggattcacactttagacaaggaatccccttttttgcacaggtgcttctggtggatggaaaaggtgtgcccatccccaataaactcttcttcatctctgtgaatgacgccaattattactccaatgcaaccaccaatgagcagggtcttgcacagttttcaatcaatactaccagtatctcggttaataaactttttgtccgggttttcactgtgcatcccaacttgtgttttcactattcatgggtagcagaagaccaccagggtgctcagcacactgcaaatcgtgttttctccttaagtggaagttacattcacctggagcctgtggctggtaccctgccctgtggccacacggagactatcacggcacactatacactgaatagacaggccatgggagagttatcggagctcagtttccattacctgatcatggctaagggagtcatcgtcagatctggaacccacactctgcctgtggagtcaggagacatgaaaggcagttttgccttatccttccctgtggagtcagacgttgcccccattgcacgaatgttcatctttgccattttaccagatggagaagttgttggagactctgaaaaatttgagattgaaaactgtctagccaacaaggtggatttgagcttcagcccagcacaaagtcccccagcctcacatgcccacctgcaagtagcagctgctccgcagtccctctgtgcccttcgtgctgtggaccaaagtgtgctgctcatgaagcctgaggctgagctctctgtgtcctcagtatataatctgctaactgtgaaggatctcaccaattttcctgacaatgtggaccagcaggaggaagaacaaggacactgtccccgtcctttcttcattcataatggagccatctatgttcccttatcaagtaatgaagcagatatttatagcttcctcaaggggatgggattgaaggtgttcactaactcaaaaatccgaaaaccaaagtcgtgttcagtcatcccttccgtgtctgcaggagcagtaggtcaaggatactatggagcaggtctaggagtagtagagagaccatatgttcctcaattaggcacatataatgtgatacccttaaataatgaacaaagttcagggccagtccctgaaacggtgcgaagctattttcctgagacttggatctgggagttggtggcagtgaactcatcaggtgtggctgaggtaggagtaacagtccctgacaccatcaccgagtggaaggcaggggccttctgcctgtccgaagatgctggacttggtatctcttccactgcctctctccgagccttccagcccttctttgtggagctcacaatgccttactctgtgattcgtggagaggtcttcacactcaaggccacggtcctaaactaccttcccaaatgcatccgggtcagtgtgcagctgaaagcctctccagccttcctagcttcccaaaatacaaagggagaagaatcctattgtatctgtggaaatgagagacaaaccttgtcttggacagtgactcctaaaactctggggaatgtgaacttctcagtgagtgcagaggcaatgcagtccttagaactctgtggaaatgaggttgttgaggtccctgagattaaaagaaaagacacagtcatcaaaaccctgttggtggaggctgaaggtattgagcaagaaaagactttcagttctatgacctgtgcctcaggtgctaatgtgtctgagcagttgtccttgaagctcccatcaaatgtggtcaaagaatctgccagagcttctttctcagttctgggtgacatattaggttctgctatgcaaaatatacaaaatctcctccagatgccatatggctgtggagaacagaacatggtcctatttgctcctaacatctatgtcttgaactatctgaatgaaacccagcagctgacgcaggagatcaaggccaaggccgttggctatctcatcactggttaccagagacagctgaactacaaacaccaagatggctcctacagcacctttggggaacgatatggcaggaaccagggcaacacttggctcacagcttttgtactgaagactttcgcccaggctcgatcctacatcttcattgatgaagcacacattacccaatctctcacgtggctctcccagatgcagaaggacaatggctgtttcaggagctctgggtcactgctcaacaatgccataaagggaggtgtagaagatgaagcgaccctctccgcctatgttactattgcccttctggaaattcctctcccagtcactaaccctattgttcgcaatgccctgttctgcctggagtcagcctggaatgtagcaaaggaggggacccatgggagccatgtctacaccaaggcattgctggcctatgctttttccctactgggaaagcaaaatcagaatagagaaatactgaactcacttgataaggaagctgtgaaagaagacaacctcgtccattgggagcgccctcagagacccaaggcaccagtggggcatctttaccaaacccaggctccctctgctgaggtggagatgacatcctatgtgctcctcgcttatctcacggcccagccagcccccacctcaggggacctgacctctgcaactaacattgtgaagtggatcatgaagcagcagaacgcccaaggtggtttctcctccacccaggacacagtggtggctctccatgccctgtccaggtatggagcagccactttcaccagaactgagaaaactgcacaggtcaccgttcaggattcacagaccttttctacaaatttccaagtagacaacaacaacctcctattactgcagcagatctcattgccagagctccctggagaatatgtcataacagtaactggggaaagatgtgtgtatcttcagacatccatgaaatacaatattcttccagagaaagaggactccccatttgctttaaaagtgcagactgtgccccaaacttgcgatggacacaaagcccacaccagctttcagatctcactgaccatcagttacacaggaaaccgtcctgcttccaatatggtgattgttgatgtaaagatggtatctggttttattcccctgaaaccaacagtaaaaatgcttgaaagatctagctctgtgagccggacagaagtgagcaacaaccatgtcctcatttatgtggaacaggtgacaaatcagacgctaagtttttccttcatggttctgcaagacatcccagtaggagacttgaagccagcaattgttaaagtctatgattactatgagacagatgagtctgtggttgctgagtatatcgccccctgcagcacagatacagagcatggaaatgtttgaggaccatacaggctgtatattttggtggattctctgtcctatacatttacttagaaggaatggagttatttgtctctataaaatagacactaaaaatatttgctgaataaatatgtacttctggtcaaactaaaaaa

SEQ ID NO: 90 - DNA, 2024 bp, HUMAN
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgaaaaaaaaaa

SEQ ID NO: 91 - DNA, 1645 bp, HUMAN
ggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctcatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtggccgtgcggcacgag

SEQ ID NO: 92 - DNA, 1312 bp, HUMAN
cagttagtacactgaaattcaaagtcatgctcataactgttaatgaaagcagattcaaagcaacaccaccaccactgaagtatttttagttatataagattggaactaccaagcatgtggctcctggtcagtgtaattctaatctcacggatatcctctgttgggggagaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgctcatatactgtcgagacagatgagtaaatatccatctggtgagagagtacgttatgaatgtaggagcccttatgaaatgtttggggatgaagaagtgatgtgtttaaatggaaactggacagaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaagatagaatcaatcataaaatgcacacctttattcagaactttagtattaaatcagttcttaatttcatttttaagtattgttttactcctttttattcatacgtaaaattttggattaatttgtgaaaatgtaattataagctgagaccggtggctctcttcttaaaagcaccatattaaaacttggaaaactaaaaaaaaaaaaaaaaa

SEQ ID NO: 93 - DNA, 1266 bp, HUMAN
tgttaatgaaagcagattcaaagcaacaccaccaccactgaagtatttttagttatataagattggaactaccaagcatgtggctcctggtcagtgtaattctaatctcacggatatcctctgttgggggagaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaagatagaatcaatcataaaatgcacacctttattcagaactttagtattaaatcagttcttaatttaatttttaagtattgttttactcctttttattcatacgtaaaattttggattaatttgtgaaaatgtaattataagctgagaccggtggctctcttcttaaaagcaccatattaaaacttggaaaactggaaaact

SEQ ID NO: 94 - DNA, 1629 bp, HUMAN
attcatgaaaatccactactccagacagacggctttggaatccaccagctacatccagctccctgaggcagagttgagaatggagagaatgttacctctcctggctctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctagagcttgccatcaagcagtggggctctcagtaaggaacttggaatgcaagctggatgcctgggtctctgggcacagcctggcccctgtgcaccgagtggccatggcatgtgtggccctgtctgcttatccttggaaggtgacagcgattccctgtgtagctctcacatgcacaggggcccatggactcttcagtctggagggtcctgggcctcctgacagcaataaataatttcgttggaaaaaaaaaaaaaaaaaaaaaaaaaaaaaaaaaaaaaaaa

SEQ ID NO: 95 - DNA, 1428 bp, HUMAN
ctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctagagcttgccatcaagcagtggggctctcagtaaggaacttggaatgcaagctggatgcctgggtctctggcacagcctggcccctgtgcaccgagtggccatggcatgtgtggccctgtctgcttatccttggaaggtgacagcgattccctgtgtagctctcacatgcacaggggcccatggactcttcagtctggagggtcctgggcctcctggaatt

SEQ ID NO: 96 - DNA, 3019 bp, HUMAN
atttgttcctgaatccttggagactgacatttttcccccctaaaggcatagacaacaaaagaaattttattgagaggaaaacacaagtccttaaactgcaaagatgtttgccaggatgtctgatctccatgttctgctgttaatggctctggtgggaaagacagcctgtgggttctccctgatgtctttattggaaagcctggacccagactggacccctgaccagtatgattacagctacgaggattataatcaggaagagaacaccagtagcacacttacccacgctgagaatcctgactggtactacactgaggaccaagctgatccatgccagcccaacccctgtgaacacggtggggactgcctcgtccatgggagcaccttcacatgcagctgcctggctcctttctctgggaataagtgtcagaaagtgcaaaatacgtgcaaggacaacccatgtggccggggccaatgtctcattacccagagtcctccctactaccgctgtgtctgtaaacacccttacacaggtcccagctgctcccaagtggttcctgtatgcaggccaaacccctgccagaatggggctacctgctcccggcataagcggagatccaagttcacctgtgcctgtcccgaccagttcaaggggaaattctgtgaaataggttctgatgactgctatgttggcgatggctactcttaccgagggaaaatgaataggacagtcaaccagcatgcgtgcctttactggaactcccacctcctcttgcaggagaattacaacatgtttatggaggatgctgaaacccatgggattggggaacacaatttctgcagaaacccagatgcggacgaaaagccctggtgctttattaaagttaccaatgacaaggtgaaatgggaatactgtgatgtctcagcctgctcagcccaggacgttgcctacccagaggaaagccccactgagccatcaaccaagcttccggggtttgactcctgtggaaagactgagatagcagagaggaagatcaagagaatctatggaggctttaagagcacggcgggcaagcacccatggcaggcgtccctccagtcctcgctgcctctgaccatctccatgccccagggccacttctgtggtggggcgctgatccacccctgctgggtgctcactgctgcccactgcaccgacataaaaaccagacatctaaaggtggtgctaggggaccaggacctgaagaaagaagaatttcatgagcagagctttagggtggagaagatattcaagtacagccactacaatgaaagagatgagattccccacaatgatattgcattgctcaagttaaagccagtggatggtcactgtgctctagaatccaaatacgtgaagactgtgtgcttgcctgatgggtcctttccctctgggagtgagtgccacatctctggctggggtgttacagaaacaggaaaagggtcccgccagctcctggatgccaaagtcaagctgattgccaacactttgtgcaactcccgccaactctatgaccacatgattgatgacagtatgatctgtgcaggaaatcttcagaaacctgggcaagacacctgccagggtgactctggaggccccctgacctgtgagaaggacggcacctactacgtctatgggatagtgagctggggcctggagtgtgggaagaggccaggggtctacacccaagttaccaaattcctgaattggatcaaagccaccatcaaaagtgaaagtggcttctaaggtactgtcttctggacctcagagcccactctccttggcaccctgacaccgggaggcctcatggccaacaatggacacctccagagcctccaggggaccacacagtagactatccctactctaagcagagacaactgccacccagcctgggccttcccagaccagcatttgcacaatatcaccaggcttcttctgcctcccttggtaacccaaggaatgatggaatcaacacaacatagtatgtttgctttccttacccaattgtaccttctagaaaatcagtgttcacagagactgcctccaccacaggcatcctgcaaatgcagactccagaatccccagcatcagcgggaaccaccatcacatctttattcctcagcccagacactcgaggcactcaacagaatcagccatccacgtctaggtatcagagaggaccacaaatacaacattctccatctgctttcagagttattattttaataaaggaagatctgggatgggctggtgggccattccagcttgccgaaatcaaagccatctgaagcctgtctctggtgaacaaacttcctctctggcctctcaggaatcagggtggacatggctcacaacagcagggccttcttctttttgacgtgcagaatctcagtggcatctgggttcacctccccactctgatgatctccagcctccactgcttctgccccccgctgctgaaatcaaacataccccaagttaaaatgaagctcccccacccccactcccggccccggttcccacaggacacgctaagaagcacagggagcatttaacaggctcaccctccctttccttttcccctcttctaccctccccaagaaaaagggccttcaaggcaggaatgagaaagcaaagccaatctctcatttagacctggcttctttcttctgaacaaagtagggttcaaaatgcagactgtcatatccagcgagtccctgaccctttctgcgaatgtaacgagcaagcagtcagcacagcctgggctgccctggcccgggattgatgtagccccggtaggtttgcctctgcagaactaatggctgtgacttcagagaaagccctgcaggaagtttaacctgcgtgtcatctgcctggtcatctcagacccatgaaattaggcgccttgtttgagctgcgtttcacacttctttagagctagctgacctttggccaaaaataaactttgaaaagaaa

SEQ ID NO: 97 - DNA, 2408 bp, HUMAN
cctgaatccttggagactgacatttttcccccctaaaggcatagacaacaaaagaaattttattgagaggaaaacacaagtccttaaactgcaaagatgtttgccaggatgtctgatctccatgttctgctgttaatggctctggtgggaaagacagcctgtgggttctccctgatgtctttattggaaagcctggacccagactggacccctgaccagtatgattacagctacgaggattataatcaggaagagaacaccagtagcacacttacccatgctgagaatcctgactggtactacactgaggaccaagctgatccatgccagcccaacccctgtgaacacggtggggactgcctcgtccatgggagcaccttcacatgcagctgcctggctcctttctctgggaataagtgtcagaaagtgcaaaatacgtgcaaggacaacccatgtggccggggccaatgtctcattacccagagtcctccctactaccgctgtgtctgtaaacacccttacacaggtcccagctgctcccaagtggttcctgtatgcaggccaaacccctgccagaatggggctacctgctcccggcataagcggagatccaagttcacctgtgcctgtcccgaccagttcaaggggaaattctgtgaaataggttctgatgactgctatgttggcgatggctactcttaccgagggaaaatgaataggacagtcaaccagcatgcgtgcctttactggaactcccacctcctcttgcaggagaattacaacatgtttatggaggatgctgaaacccatgggattggggaacacaatttctgcagaaacccagatgcggacgaaaagccctggtgctttattaaagttaccaatgacaaggtgaaatgggaatactgtgatgtctcagcctgctcagcccaggacgttgcctacccagaggaaagccccactgagccatcaaccaagcttccggggtttgactcctgtggaaagactgagatagcagagaggaagatcaagagaatctatggaggctttaagagcacggcgggcaagcacccatggcaggcgtccctccagtcctcgctgcctctgaccatctccatgccccagggccacttctgtggtggggcgctgatccacccctgctgggtgctcactgctgcccactgcaccgacataaaaaccagacatctaaaggtggtgctaggggaccaggacctgaagaaagaagaatttcatgagcagagctttagggtggagaagatattcaagtacagccactacaatgaaagagatgagattccccacaatgatattgcattgctcaagttaaagccagtggatggtcactgtgctctagaatccaaatacgtgaagactgtgtgcttgcctgatgggtcctttccctctgggagtgagtgccacatctctggctggggtgttacagaaacaggaaaagggtcccgccagctcctggatgccaaagtcaagctgattgccaacactttgtgcaactcccgccaactctatgaccacatgattgatgacagtatgatctgtgcaggaaatcttcagaaacctgggcaagacacctgccagggtgactctggaggccccctgacctgtgagaaggacggcacctactacgtctatgggatagtgagctggggcctggagtgtgggaagaggccaggggtctacacccaagttaccaaattcctgaattggatcaaagccaccatcaaaagtgaaagtggcttctaaggtactgtcttctggacctcagagcccactctccttggcaccctgacaccgggaggcctcatggccaacaatggacacctccagagcctccaggggaccacacagtagactatcctactctaagcagagacaactgccacccagcctgggccttcccagaccagcatttgcacaatatcaccaggcttcttctgcctcccttggtaacccaaggaatgatggaatcaacacaacatagtatgtttgctttccttacccaattgtaccttctagaaaatcagtgttcacagagactgcctccaccacaggcatcctgcaaatgcagactccagaatccccagcatcagcgggaaccaccatcacatctttattcctcagcccagacactcgaggcactcaacagaatcagccatccacgtctaggtatcagagaggaccacaaatacaacattctccatctgctttcagagttattattttaataaaggaagatctgggatgggctggtgggccattccagcttgccgaaatcaagccatctgaagcctgtctctggtgaacaaacttcctctctggcctctcaggaatcagggtggcatggctcacaacagcagggccttcttcttttt

SEQ ID NO: 98 - DNA, 2211 bp, HUMAN
gcagttgagctgaatgaatacctccgaagccgctttgttctccagatgtgaatagctccactataccagcctcgtcttccttccgggggacaacgtgggtcagggcacagagagatatttaatgtcaccctcttggggctttcatgggactccctctgccacattttttggaggttgggaaagttgctagaggcttcagaactccagcctaatggatcccaaactcgggagaatggctgcgtccctgctggctgtgctgctgctgctgctggagcgcggcatgttctcctcaccctccccgcccccggcgctgttagagaaagtcttccagtacattgacctccatcaggatgaatttgtgcagacgctgaaggagtgggtggccatcgagagcgactctgtccagcctgtgcctcgcttcagacaagagctcttcagaatgatggccgtggctgcggacacgctgcagcgcctgggggcccgtgtggcctcggtggacatgggtcctcagcagctgcccgatggtcagagtcttccaatacctcccgtcatcctggccgaactggggagcgatcccacgaaaggcaccgtgtgcttctacggccacttggacgtgcagcctgctgaccggggcgatgggtggctcacggacccctatgtgctgacggaggtagacgggaaactttatggacgaggagcgaccgacaacaaaggccctgtcttggcttggatcaatgctgtgagcgccttcagagccctggagcaagatcttcctgtgaatatcaaattcatcattgaggggatggaagaggctggctctgttgccctggaggaacttgtggaaaaagaaaaggaccgattcttctctggtgtggactacattgtaatttcagataacctgtggatcagccaaaggaagccagcaatcacttacggaacccgggggaacagctacttcatggtggaggtgaaatgcagagaccaggattttcactcaggaacctttggtggcatccttcatgaaccaatggctgatctggttgctcttctcggtagcctggtagactcgtctggtcatatcctggtccctggaatctatgatgaagtggttcctcttacagaagaggaaataaatacatacaaagccatccatctagacctagaagaataccggaatagcagccgggttgagaaatttctgttcgatactaaggaggagattctaatgcacctctggaggtacccatctctttctattcatgggatcgagggcgcgtttgatgagcctggaactaaaacagtcatacctggccgagttataggaaaattttcaatccgtctagtccctcacatgaatgtgtctgcggtggaaaaacaggtgacacgacatcttgaagatgtgttctccaaaagaaatagttccaacaagatggttgtttccatgactctaggactacacccgtggattgcaaatattgatgacacccagtatctcgcagcaaaaagagcgatcagaacagtgtttggaacagaaccagatatgatccgggatggatccaccattccaattgccaaaatgttccaggagatcgtccacaagagcgtggtgctaattccgctgggagctgttgatgatggagaacattcgcagaatgagaaaatcaacaggtggaactacatagagggaaccaaattatttgctgcctttttcttagagatggcccagctccattaatcacaagaaccttctagtctgatctgatccactgacagattcacctcccccacatccctagacagggatggaatgtaaatatccagagaatttgggtctagtatagtacattttcccttccatttaaaatgtcttgggatatctggatcagtaataaaatatttcaaaggcacagatgttggaaatggtttaaggtcccccactgcacaccttcctcaagtcatagctgcttgcagcaacttgatttccccaagtcctgtgcaatagccccaggattggattccttcaaaccttttagcatatctccaaccttgcaatttgattggcataatcactccagtttgctttctaggtcctcaagtgctcgtgacacataatcattccatccaatgatcgcctttgctttaccactctttccttttatcttattaataaaaatgttggtctccaccactgactacaaaaaaaaaaaaaaa

SEQ ID NO: 99 - DNA, 1624 bp, HUMAN
ggttgggaaagttgctagaggcttcagaactccagcctaatggatcccaaactcgggagaatggctgcgtccctgctggctgtgctgctgctgctgctggagcgcggcatgttctcctcaccctccccgcccccggcgctgttagagaaagtcttccagtacattgacctccatcaggatgaatttgtgcagacgctgaaggagtgggtggccatcgagagcgactctgtccagcctgtgcctcgcttcagacaagagctcttcagaatgatggccgtggctgcggacacgctgcagcgcctgggggcccgtgtggcctcggtggacatgggtcctcagcagctgcccgatggtcagagtcttccaatacctcccgtcatcctggccgaactggggagcgatcccacgaaaggcaccgtgtgcttctacggccacttggacgtgcagcctgctgaccggggcgatgggtggctcacggacccctatgtgctgacggaggtaggcgggaaactttatggacgaggagcgaccgacaacaaaggccctgtcttggcttggatcaatgctgtgagcgccttcagagccctggagcaagatcttcctgtgaatatcaaattcatcattgaggggatggaagaggctggctctgttgccctggaggaacttgtggaaaaagaaaaggaccgattcttctctggtgtggactacattgtaatttcagataacctgtggatcagccaaaggaagccagcaatcacttacggaacccgggggaacagctacttcatggtggaggtgaaatgcagagaccaggattttcactcaggaacctttggtggcatccttcatgaactaatggctgatctggttgctcttctcggtagcctggtagactcgtctggtcatatcctggtccctggaatctatgatgaagtggttcctcttacagaagaggaaataaatacatacaaagccatccatctagacctagaagaataccggaatagcagccgggttgagaaatttctgttcgatactaaggaggagattctaatgcacctctggaggtacccatctctttctattcatgggatcgagggcgcgtttgatgagcctggaactaaaacagtcatacctggccgagttataggaaaattttcaatccgtctagtccctcacatgaatgtgtctgcggtggaaaaacaggtgacacgacatcttgaagatgtgttctccaaaagaaatagttccaacaagatggttgtttccatgactctaggactacacccgtggattgcaaatattgatgacacccagtatctcgcagcaaaaagagcgatcagaacagtgtttggaacagaaccagatatgatccgggatggatccaccattccaattgccaaaatgttccaggagatcgtccacaagagcgtggtgctaattccgctgggagctgttgatgatggagaacattcgcagaatgagaaaatcaacaggtggaactacatagagggaaccaaattatttgctgcctttttcttagagatggcccagctccattaatcacaagaaccttctagtctgatctgatccactgacagattcacctcccccacatccctag

SEQ ID NO: 100 - DNA, 1043 bp, HUMAN
tgttaatgaaagcagattcaaagcaacaccaccaccactgaagtatttttagttatataagattggaactaccaagcatgtggctcctggtcagtgtaattctaatctcacggatatcctctgttgggggagaagcaatgttctgtgattttccaaaaataaaccatggaattctatatgatgaagaaaaatataagccattttcccaagttcctacaggggaagttttctattactcctgtgaatataattttgtgtctccttcaaaatccttttggactcgcataacgtgcgcagaagaaggatggtcaccaacaccaaagtgtctcagactgtgtttctttccttttgtggaaaatggtcattctgaatcttcaggacaaacacatctggaaggtgatactgtacaaattatttgcaacacaggatacagacttcaaaacaatgagaacaacatttcatgtgtagaacggggctggtccactcctcccaaatgcaggtccactatttctgcagaaaaatgtgggccccctccacctattgacaatggagacattacttcattcctgttgtcagtatatgctccaggttcatcagttgagtaccagtgccagaacttgtatcaacttgagggtaacaatcaaataacatgtagaaacggacaatggtcagaaccaccaaaatgcttagatccatgtgtaatatcacaagaaattatggaaaaatataacataaaattaaagtggacaaaccaacaaaagctttattcaagaacaggtgacatagttgaatttgtttgtaaatctggatatcatccaacaaaatctcattcatttcgagcaatgtgtcagaatgggaaactggtatatcccagttgtgaagaaaaatagaatcaatggcattactattagtaaaatgcacacctttttctgaatttactattatatttgttttcaatttcatttttcaagtactgttttactcatttttattcataaataaagttttgtgttgatttgtgaaaatgcaattacaagagccaa

SEQ ID NO: 101 - DNA, 1040 bp, HUMAN
ggaattcggcacgagattcaaagcaacaccaccaccactgaagtatttttagttatataagattggaactaccaagcatgtggctcctggtcagtgtaattctaatctcacggatatcctctgttgggggagaagcaatgttctgtgattttccaaaaataaaccatggaattctatatgatgaagaaaaatataagccattttcccaagttcctacaggggaagttttctattactcctgtgaatataattttgtgtctccttcaaaatccttttggactcgcataacgtgcgcagaagaaggatggtcaccaacaccaaagtgtctcagactgtgtttctttccttttgtggaaaatggtcattctgaatcttcaggacaaacacatctggaaggtgatactgtacaaattatttgcaacacaggatacagacttcaaaacaatgagaacaacatttcatgtgtagaacggggctggtccactcctcccaaatgcaggtccactatttctgcagaaaaatgtgggccccctccacctattgacaatggagacattacttcattcctgttgtcagtatatgctccaggttcatcagttgagtaccagtgccagaacttgtatcaacttgagggtaacaatcaaataacatgtagaaacggacaatggtcagaaccaccaaaatgcttagatccatgtgtaatatcacaagaaattatggaaaaatataacataaaattaaagtggacaaaccaacaaaagctttattcaagaacaggtgacatagttgaatttgtttgtaaatctggatatcatccaacaaaatctcattcatttcgagcaatgtgtcagaatgggaaactggtatatcccagttgtgaggaaaaatagaatcaatggcattactattagtaaaatgcacacctttttctgaatttactattatatttgttttcaatttcatttttcaagtactgttttactcatttttattcataaataaagttttgtgttgatttgtgaaaatgcaattacaaaaaa

SEQ ID NO: 102 - DNA, 570 bp, HUMAN
agggacccgcagctcagctacagcacagatcagcaccatgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccatggtgcggaggactcgctggccgatcaggctgccaataaatggggcaggagtggcagagaccccaatcacttccgacctgctggcctgcctgagaaatactgagcttcctcttcactctgctctcaggagacctggctatgaggccctcggggcagggatacaaagttagtgaggtctatgtccagagaagctgagatatggcatataataggcatctaataaatgcttaagaggtcaaaaaaaaaaaaaaaaaaaaaaaaaaaaaa

SEQ ID NO: 103 - DNA, 369 bp, HUMAN
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgtctgggctgcagaagcgatcagcgatgccagagagaatatccagagattctttggccatggtgcggaggactcgctggctgatcaggctgccaatgaatggggcaggagtggcaaagaccccaatcacttccgacctgctggcctgtctgagaaatactga

SEQ ID NO: 104 - DNA, 1485 bp, HUMAN
ggtgggaatggcaggctgcgtcccactgctccagggcctggtcctggtcctcgccctccatcgtgtggagccctcagtatttctcccggcctccaaagcaaacgacgttctggtgaggtggaagcgtgcgggctcctatcttctggaagaactcttcgagggaaacttggaaaaagaatgttatgaagaaatctgtgtctatgaagaagcaagagaagtgtttgaaaatgaagtagtcactgatgaattctggagacgatataagggcggctccccgtgcatctcccagccctgcctccacaacggctcttgccaggacagcatctggggctacacctgcacctgctcccccggctatgagggcagcaactgcgagctggctaaaaatgaatgtcacccagagcggactgatgggtgtcaacacttctgcctcccaggacaggaatcctacacgtgcagctgtgctcagggctacaggcttggtgaggaccacaaacagtgtgtgccccacgaccagtgtgcctgcggggtgctgacctctgagaagcgtgcaccggatctacaggacctcccgtggcaggtaaagttaacaaattccgaaggaaaagacttctgtggtggtgttataatacgggaaaattttgtactgacaacagcaaaatgttcactgttacacaggaatattactgtaaaaacatattttaacagaacgagccaagacccgctgatgatcaagataacgcacgtccatgtgcacatgcggtatgacgcggacgcgggggagaatgacctgtcactgctggagctggagtggcccatccagtgcccaggtgcggggctccccgtgtgcacccctgagaaagacttcgctgagcacctcctcatcccacgcaccaggggcctcctcagcggctgggcacgcaatggcactgacctgggcaactcgctgaccacgcggcctgtcacacttgtggagggggaggagtgcgggcaggtcctgaatgtgactgtcaccaccaggacctactgtgagagaagcagcgtggcggccatgcactggatggatggaagtgtggtcaccagagaacacagaggctcctggtttctcacgggggtcctgggctcgcagccagtaggagggcaggctcacatggtccttgtcaccaaggtctccaggtactcactctggtttaaacagatcatgaactaactgaaactcagctagccagaatgaacaacacaaccggaagcgggattccaagctggcactgccactgtggagggcgctgaaacttcatcacacactgagaggccgtcacagccccagaccacccgcttggcccacgcagcagcagagccgccgtttgctgggttgtttaccgagcactgtgacctttctttccctggaactctttatctcaatagagaccttaaaagaaaacatgagatacgttaaataataaaataagataatctgtcagtcat

SEQ ID NO: 105 - DNA, 1201 bp, HUMAN
agtcgccgctgggcaccccgagaccatggggaagctcgtggcgctggtcctgctgggggtcggcctgtccttagtcggggagatgttcctggcgtttagagaaagggtgaatgcctctcgagaagtggagccagtagaacctgaaaactgccaccttattgaggaacttgaaagtggctctgaagatattgatatacttcctagtgggctggcttttatctccagtggattaaaatatccaggcatgccaaactttgcgccagatgaaccaggaaaaatcttcttgatggatctgaatgaacaaaacccaagggcacaagcgctagaaatcagtggtggatttgacaaagaattatttaatccacatgggatcagtattttcatcgacaaagacaatactgtgtatctttatgttgtgaatcatccccacatgaagtccactgtggagatatttaaatttgaggaacaacaacgttctctggtatacctgaaaactataaaacatgaacttctcaaaagtgtgaatgacattgtggttcttggaccagaacagttctatgccaccagagaccactattttaccaactccctcctgtcattttttgagatgatcttggatcttcgctggacttatgttcttttctacagcccaagggaggttaaagtggtggccaaaggattttgtagtgccaatgggatcacagtctcagcagaccagaagtatgtctatgtagctgatgtagcagctaagaacattcacataatggaaaaacatgataactgggatttaactcaactgaaggtgatacagttgggcaccttagtggataacctgactgtcgatcctgccacaggagacattttggcaggatgccatcctaatcctatgaagctactgaactataaccctgaggaccctccaggatcagaagtacttcgcatccagaatgttttgtctgagaagcccagggtgagcaccgtgtatgccaacaatggctctgtgcttcagggcacctctgtggcttctgtgtaccatgggaaaattctcataggcaccgtatttcacaaaactctgtactgtgagctctagactctagatagtaaaaaaaaaaaaaaaaagtctacatattttgtaaaagtaaactgataattgtatgataagtggcactgtaagtaaatagcaaacaccaaaaaaaaaaaa

SEQ ID NO: 106 - DNA, 1064 bp, HUMAN
atggggaagctcgtggcgctggtcctgctgggggtcggcctgtccttagtcggggagatgttcctggcgtttagagaaagggtgaatgcctctcgagaagtggagccagtagaacctgaaaactgccaccttattgaggaacttgaaagtggctctgaagatattgatatattcctcagtgggctggcttttatctccagtggattaaaatatccaggcatgccaaactttgcgccagatgaaccaggaaaaatcttcttgatggatctgaatgaacaaaacccaagggcacaagcactagaaatcagtggtggatttgacaaagaattatttaatccacatgggatcagtattttcatcgacaaagacaatactgtgtatctttatgttgtgaatcatccccacatgaagtccactgtggagatatttaaatttgaggaacaacaacgttctctggtatacctgaaaactataaaacatgaacttctcaaaagtgtgaatgacattgtggttcttggaccagaacagttctatgccaccagagaccactattttaccaactccctcctgtcattttttgagatgatcttggatcttcgctggacttatgttcttttctacagcccaagggaggttaaagtggtggccaaaggattttgtagtgccaatgggatcacagtctcagcagaccagaagtatgtctatgtagctgatgtagcagctaagaacattcacataatggaaaaacatgataactgggatttaactcaactgaaggtgatacagttgggcaccttagtggataacctgactgtcgatcctgccacaggagacattttggcaggatgccatcctaatcctatgaagctactgaactataaccctgaggaccctccaggatcagaagtacttcgcatccagaatgttttgtctgagaagcccagggtgagcaccgtgtatgccaacaatggctctgtgcttcagggcacctctgtggcttctgtgtaccatgggaaaattctcataggcaccgtatttcacaaaactctgtactgtgagctcta

SEQ ID NO: 107 - DNA, 767 bp, HUMAN
gccgccccgcgagaagaagagcgggaagaggcggacagcgaggccaagatttcagctgcgggacggtcaggggagacctccaggcgcagggaaggacggccagggtgacacggaagcatgcgacggctgctgatccctctggccctgtggctgggtgcggtgggcgtgggcgtcgccgagctcacggaagcccagcgccggggcctgcaggtggccctggaggaatttcacaagcacccgcccgtgcagtgggccttccaggagaccagtgtggagagcgccgtggacacgcccttcccagctggaatatttgtgaggctggaatttaagctgcagcagacaagctgccggaagagggactggaagaaacccgagtgcaaagtcaggcccaatgggaggaaacggaaatgcctggcctgcatcaaactgggctctgaggacaaagttctgggccggttggtccactgccccatagagacccaagttctgcgggaggctgaggagcaccaggagacccagtgcctcagggtgcagcgggctggtgaggacccccacagcttctacttccctggacagttcgccttctccaaggccctgccccgcagctaagccagcactgagatgcgtggtgcctccaggaccgctgcgggtggtaaccagtggaagaccccagcccccagggagaggaacccgttctatccccagccatgataataaagctgctctcccagctgcctctcaaaaaaaaaaaaaaaaaaaaaaaaaaa

SEQ ID NO: 108 - DNA, 695 bp, HUMAN
aagatttcagctgcgggacggtcaggggagacctccaggcgcagggaaggacggccagggtgacacggaagcatgcgacggctgctgatccctctggccctgtggctgggcgcggtgggcgtgggcgtcgccgagctcacggaagcccagcgccggggcctgcaggtggccctggaggaatttcacaagcacccgcccgtgcagtgggccttccaggagaccagtgtggagagcgccgtggacacgcccttcccagctggaatatttgtgaggctggaatttaagctgcagcagacaagctgccggaagagggactggaagaaacccgagtgcaaagtcaggcccaatgggaggaaacggaaatgcctggcctgcatcaaactgggctctgaggacaaagttctgggccggttggtccactgccccatagagacccaagttctgcgggaggctgaggagcaccaggagacccagtgcctcagggtgcagcgggctggtgaggacccccacagcttctacttccctggacagttcgccttctccaaggccctgccccgcagctaagccagcactgagctgcgtggtgcctccaggaccgctgccggtggtaaccagtggaagaccccagcccccagggagaggaccccgttctatccccagccatgataataaagctgctctcccagctgcctctc

SEQ ID NO: 109 - DNA, 1505 bp, HUMAN
cagagtctccgcttaggcggcccgtgggggtcggggtcgcggtcgcggtcgcggaccggggacagagcaatggggcgggcgcgcagcgccggctgcactcccagagcctcccaccgccgccccacggccgcctagcgcgcgccgggactgcatttcccataggcccacgctgcggagcaccgcccacccgagtctcacgcgagtgcgctgccgcgaggtgtccctgccgcagcccccgcccgcccgcagagcttttgaaaggcggcgggaggcggcgagcgccatggccagtccgggctgcctgctgtgcgtgctgggcctgctactctgcggggcggcgagcctcgagctgtctagaccccacggcgacaccgccaagaagcccatcatcggaatattaatgcaaaaatgccgtaataaagtcatgaaaaactatggaagatactatattgctgcgtcctatgtaaagtacttggagtctgcaggtgcgagagttgtaccagtaaggctggatcttacagagaaagactatgaaatacttttcaaatctattaatggaatccttttccctggaggaagtgttgacctcagacgctcagattatgctaaagtggccaaaatattttataacttgtccatacagagttttgatgatggagactattttcctgtgtggggcacatgccttggatttgaagagctttcactgctgattagtggagagtgcttattaactgccacagatactgttgacgtggcaatgccgctgaacttcactggaggtcaattgcacagcagaatgttccagaattttcctactgagttgttgctgtcattagcagtagaacctctgactgccaatttccataagtggagcctctccgtgaagaattttacaatgaatgaaaagttaaagaagtttttcaatgtcttaactacaaatacagatggcaagattgagtttatttcaacaatggaaggatataagtatccagtatatggtgtccagtggcatccagagaaagcaccttatgagtggaagaatttggatggcatttcccatgcacctaatgctgtgaaaaccgcattttatttagcagagttttttgttaatgaagctcggaaaaacaaccatcattttaaatctgaatctgaagaggagaaagcattgatttatcagttcagtccaatttatactggaaatatttcttcatttcagcaatgttacatatttgattgaaagtcttcaatttgttaacagagcaaatttgaataattccatgattaaactgttagaataacttgctactcatggcaagattaggaagtcacagattcttttctataatgtgcctggctctgattcttcattctgtatgtgactatttatataacattagataattaaatagtgagacataaatagagtgtttttcatggaaaagccttcttatatctgaagattgaaaaaaataaatttactgaaatacaaaaaaaaaaaaaaa

SEQ ID NO: 110 - DNA, 1280 bp, HUMAN
tgccgcagcccccgcccgcccgcagagcttttgaaaggcggcgggaggcggcgagcgccatggccagtccgggctgcctgctgtgcgtgctgggcctgctactctgcggggcggcgagcctcgagctgtctagaccccacggcgacaccgccaagaagcccatcatcggaatattaatgcaaaaatgccgtaataaagtcatgaaaaactatggaagatactatattgctgcgtcctatgtaaagtacttggagtctgcaggtgcgagagttgtaccagtaaggctggatcttacagagaaagactatgaaatacttttcaaatctattaatggaatccttttccctggaggaagtgttgacctcagacgctcagattatgctaaagtggccaaaatattttataacttgtccatacagagttttgatgatggagactattttcctgtgtggggcacatgccttggatttgaagagctttcactgctgattagtggagagtgcttattaactgccacagatactgttgacgtggcaatgccgctgaacttcactggaggtcaattgcacagcagaatgttccagaattttcctactgagttgttgctgtcattagcagtagaacctctgactgccaatttccataagtggagcctctccgtgaagaattttacaatgaatgaaaagttaaagaagtttttcaatgtcttaactacaaatacagatggcaagattgagtttatttcaacaatggaaggatataagtatccagtatatggtgtccagtggcatccagagaaagcaccttatgagtggaagaatttggatggcatttcccatgcacctaatgctgtgaaaaccgcattttatttagcagagttttttgttaatgaagctcggaaaaacaaccatcattttaaatctgaatctgaagaggagaaagcattgatttatcagttcagtccaatttatactggaaatatttcttcatttcagcaatgttacatatttgattgaaagtcttcaatttgttaacagagcaaatttgaataattccatgattaaactgttagaataacttgctactcatggcaagattaggaagtcacagattcttttctataatgtgcctggctctgattcttcattatgtatgtgactatttatataacattagataattaaatagtgagacataaatagagtgctttttcatggaaaagccttcttatatctgaagattgaaaaataaatttactgaaatacaaaaaaaaaaaaaaa

SEQ ID NO: 111 - DNA, 5059 bp, HUMAN
aaactcatctatcctttacggcaagggtacctacggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaattcggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccactcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctgtgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaacgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 112 - DNA, 5041 bp, HUMAN
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 113 - DNA, 3332 bp, HUMAN
acttggctctcgccctccggccgggaagcatggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccggggcgtggtcatcgtggctgtgattgtgtgcatcctggtcctggcggtgctgggcgctgtcctctatttcctctataagaagggcaagctgccgtgcaggcgctcagggaagcaggagatcacgctacccccgtctcgtaagagcgaacttgtagttgaagttaagtcagataagctcccagaagagatgggcctcctgcagggcagcagcggtgacaagagggctccgggagaccagggagagaaatacatcgatctgaggcattagccccgaatcacttcagctcccttccctgcctggaccattcccagctccctgctcactcttctctcagccaaagcctccaaagggactagagagaagcctcctgctcccctcgcctgcacaccccctttcagagggccactgggttaggacctgaggacctcacttggccctgcaaggcccgcttttcagggaccagtccaccaccatctcctccacgttgagtgaagctcatcccaagcaaggagccccagtctcccgagcgggtaggagagtttcttgcagaacgtgttttttctttacacacattatggctgtaaatacctggctcctgccagcagctgagctgggtagcctctctgagctggtttcctgccccaaaggctggcttccaccatccaggtgcaccactgaagtgaggacacaccggagccaggcgcctgctcatgttgaagtgcgctgttcacacccgctccggagagcaccccagcagcatccagaagcagctgcagtgttgctgccaccaccctcctgtctgcctcttcaaagtctcctgtgacattttttctttggtcagaagccaggaactggtgtcattccttaaaagatacgtgccggggccaggtgtggtggctcacgcctgtaatcccagcactttgggaggccgaggcgggcggatcacaaagtcaggacgagaccatcctggctaacacggtgaaaccctgtctctactaaaaatacaaaaaaaaattagctaggcgtagtggttggcacctatagtcccagctactcggaaggctgaagcaggagaatggtatgaatccaggaggtggagcttgcagtgagccgagaccgtgccactgcactccagcctgggcaacacagcgagactccgtctcgaggaaaaaaaaagaaaagatacgtgcctgcggtgaggaagctgggcgctgttttcgagttcaggtgaattagcctcaatcccccgtgttcacttggctcccatagccctcttgatggatcacgtaaaactgaaaggcagcggggagcagacaaagatgaggtctacactgtccttcatggggattaaagctatggttatattagcaccaaacttctacaaaccaagctcagggccccaaccctagaagggcccaaatgagagaatggtacttagggatggaaaacgggcctggctagagcttcgggtgtgtgtgtctgtctgtgtgtatgcatacatatgtgtgtatatatggttttgtcaggtgtgtaaatttgcaaattgtttcctttatatatgtatgtatatatatatatgaaaatatatatatatatgaaaaataaagcttaattgtcccagaaatcata

SEQ ID NO: 114 - DNA, 2943 bp, HUMAN
gggaagcatggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcaggcctggaacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacagaccaggtgctggaaagggggcctgtgcttcagttgcatgacctgaaacgggaggcaggaggcggctatcgctgcgtggcgtctgtgcccagcatacccggcctgaaccgcacacagctggtcaagctggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccggggcgtggtcatcgtggctgtgattgtgtgcatcctggtcctggcggtgctgggcgctgtcctctatttcctctataagaagggcaagctgccgtgcaggcgctcagggaagcaggagatcacgctgcccccgtctcgtaagaccgaacttgtagttgaagttaagtcagataagctcccagaagagatgggcctcctgcagggcagcagcggtgacaagagggctccgggagaccagggagagaaatacatcgatctgaggcattagccccgaatcacttcagctcccttccctgcctggaccattcccagctccctgctcactcttctctcagccaaagctcaaagggactagagagaagcctcctgctcccctcgcctgcacaccccctttcagagggccactgggttaggacctgaggacctcacttggccctgcaaggcccgcttttcagggaccagtccaccaccatctcctccacgttgagtgaagctcatcccaagcaaggagccccagtctcccgagcgggtaggagagtttcttgcagaacgtgttttttctttacacacattatgctgtaaatacgctcgtcctgccagcagctgagctgggtagcctctctgagctggtttcctgccccaaaggctggcattccaccatccaggtgcaccactgaagtgaggacacaccggagccaggcgcctgctcatgttgaagtgcgctgttcacacccgctccggagagcaccccagcagcatccagaagcagctgcagtgcaagcttgcatgcctgcgtgttgctgcaccaccctcctgtctgcctcttcaaagtctcctgtgacattttttctttggtcagaggccaggaactgtgtcattccttaaagatacgtgccggggccaggtgtggctcacgcctgtaatcccagcactttgggaggccgaggcggcggatcacaaagtcagacgagaccatcctggctaacacggtgaaaccctgtctctactaaaaatacaaaaaaaaattagctaggcgtagtggttggcacctatagtcccagctactcggaaggctgaagcaggagaatggtatgaatccaggaggtggagcttgcagtgagccgagaccgtgccactgcactccagcctgggcaacacagcgagactccgtctcgagccggccggttgcgcgggccctcggaccctcagagaggcgagggttcgagggcacgagttcgaggccaacctggtccacatgggttg

SEQ ID NO: 115 - DNA, 8272 bp, HUMAN
gcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcaggttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacagaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggacagcccaggcagtacaatgtgggtccctctgtctccaagtacccactgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccattgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagagacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccactattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggagttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacatccccagggaagatgtagactatcacctgtacccacacggtccgggactcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttatgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccacaacttgaagttcatctatttgatataagacaccttcgggggaaataattcctgtgaatattctttttcaattcagcaaacatttgaaaatctatgatgtgcaagtctaattgttgatttcagtacaagattttctaaatcagttgctacaaaaactgattggtttttgtcacttcatctcttcactaatggagatagctttacactttctgctttaatagatttaagtggaccccaatatttattaaaattgctagtttaccgttcagaagtataatagaaataatctttagttgctcttttctaaccattgtaattcttcccttcttccctccacctttccttcattgaataaacctctgttcaaagagattgcctgcaagggaaataaaaatgactaagatattaaaaaaaaaaaaaaaaaa

SEQ ID NO: 116 - DNA, 7680 bp, HUMAN
gaagagcaagaggcaggctcagcaaatggttcagccccagtccccggtggctgtcagtcaaagcaagcccggttgttatgacaatggaaaacactatcagataaatcaacagtgggagcggacctacctaggtaatgtgttggtttgtacttgttatggaggaagccgaggttttaactgcgaaagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggttgaagacacaaggaaataagcaaatgctttgcacgtgcctgggcaacggagtcagctgccaagagacagctgtaacccagacttacggtggcaacttaaatggagagccatgtgtcttaccattcacctacaatggcaggacgttctactcctgcaccacggaagggcgacaggacggacatctttggtgcagcacaacttcgaattatgagcaggaccagaaatactctttctgcacagaccacactgttttggttcagactcaaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcaacttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggccagcccaggcagtacaatgtgggtccctctgtctccaagtaccccctgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccatcgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggtgttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttacgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccaca